SEQUENCE LISTING (5 sequences)

SEQ ID NO: 1 - DNA, 1369 bp, Homo sapiens
gtgtgccggatttggttagctgagcccaccgagagggtaagtgacagctgctcctgcgcttgccatggcaccagcggggaggctggggtcaaggctgagcctccatccctgtcccccacatggggggacaggggtccaggtccaggggcagatcctactccctccatgggccggatcttccccacagggcagggctgatccagctgtgggtctcttggttccctctttcagcgcctgcaggatgaaagctctctgtctcctcctcctccctgtcctggggctgttggtgtctagcaagaccctgtgctccatggaagaagccatcaatgagaggatccaggaggtcgccggctccctaagtgaggaccccccacttgggcaagctccccaagggtctcagagacctcactgatccctggcacagacctgactccaacccagccccagcgctcaccaaatctcatcctcaaatccaaccagatcataaattcaaccccaactccactcccaacccctccgactgtccccaccttatccacggctccaaacccaatccccgctctcactccaaaccttcccttactccaaaacacccaactcaagacagggtcctggaggccagtgagctcctatgcccacagggacctagctccaaaccaacagggctaggggaggatgggggagggaccgtttggtctcacagctccccctgtctcctttcctcctgccccccagtatttagggcaataagcagcattggcctggagtgccagagcgtcacctccaggggggacctggctacttgcccccgaggtgagtgcaggagactgttgtccaggcgcccatttctgttccaagtcccctgggaatgccccctccccgccacgttccccgtgtccagcctctactcccctaggatcttggtcctgactcccagccttctccgcccaccatctggacactggtgtccaccctcactccctgcctccagtgcccattcagtggttggagcctccagccgtccccgtccccacccccgcccccccaacccccctccgcgctccccacccccctcccgctcccaccctcagcctcccagctcagagtccacgctcctgtgttccgggctgcaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatg

SEQ ID NO: 2 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 3 - DNA, 21 bp, Artificial
  primer
gtcaaccccaccgtgttcttc

SEQ ID NO: 4 - DNA, 23 bp, Artificial
  primer
tttctgctgtctttgggaccttg

SEQ ID NO: 5 - DNA, 21 bp, Artificial
  primer
ccctaatatttagggcaataa

(19) United States
(12) Patent Application Publication
(10) Pub. No.: US 2013/0252987 A1
(43) Pub. Date: Sep. 26, 2013

(54) METHOD OF REGULATING PLASMA LIPOPROTEINS

(71) Applicant: Shirya Rashid, Burlington (CA)

(72) Inventor: Shirya Rashid, Burlington (CA)

(73) Assignee: MCMASTER UNIVERSITY, Hamilton, ON (CA)

(21) Appl. No.: 13/876,120

(22) PCT Filed: Oct. 12, 2011
(86) PCT No.: PCT/CA2011/001150

§ 371 Date: Mar. 26, 2013

Related U.S. Application Data
(60) Provisional application No. 61/392,089, filed on Oct. 12, 2010.
(60) Provisional application No. 61/422,250, filed on Dec. 13, 2010.

(51) Int. Cl. C12N 5/071

(52) U.S. Cl. CPC C12N 5/0602
USPC 514/275; 435/366; 435/6.12; 435/11; 435/29

(57) ABSTRACT

A method of modulating the level of lipoproteins in human cells comprising the step of inhibiting resistin in the cells or cellular environment. The method is useful to treat cardiovascular disease.

### FIELD OF THE INVENTION

The present invention relates generally to a method of regulating plasma lipoproteins, and more particularly, to a method in which resistin is inhibited to regulate lipoprotein levels.

### BACKGROUND OF THE INVENTION

The worldwide prevalence of obesity has reached epidemic proportions, with over 1 billion individuals worldwide characterized as being obese. In North America alone, 1 in 3 adults are obese. This is a problem because obese individuals are at increased risk of developing metabolic disorders, with associated high morbidity and mortality rates. In particular, obese individuals are at greatly elevated risk of developing atherosclerotic cardiovascular disease (ASCVD), the leading cause of death in North America.

Fundamental to the accelerated rate of ASCVD development in obese individuals is the increased presence of dyslipidemia in obesity. Dyslipidemia is highly prevalent in obese individuals, with up to 60% of abdominally obese individuals having dyslipidemia. The characteristic dyslipidemia of obesity is an elevation in plasma levels of triglycerides, a reduction in plasma high-density lipoprotein (HDL) cholesterol, and an increase in plasma numbers of low-density-lipoprotein (LDL) particles, which are small and dense. These lipid and lipoprotein abnormalities are collectively termed the lipid triad, the presence of which is strongly correlated with ASCVD. The primary lipoprotein abnormality that drives the development of the lipid triad in obesity is an elevation in plasma levels of very-low-density-lipoprotein (VLDL), which precedes and is metabolically linked to each component of the lipid triad.

Elevated VLDL in obesity is due primarily to increased hepatic secretion of VLDL triglycerides and apolipoprotein (apo) B. While several mechanisms have been proposed to account for increased hepatic VLDL secretion in obesity, including peripheral insulin resistance and increased free fatty acid flux to the liver, these factors only partially explain increased hepatic VLDL secretion in obesity and do so in some but not all in vivo or cell culture models. Therefore, other factors need to be investigated to more comprehensively understand dyslipidemia development in obesity.

Such other factors may originate at adipose tissue, which is in excess in obesity. Adipose tissue plays an important role in regulating systemic metabolism via inter-tissue communication to metabolically active tissues, including the liver. Such cross-talk is mediated by adipocyte-derived factors (adipokines) and cytokines secreted by adipose tissue. These secreted signaling molecules are potential therapeutic targets for prevention or treatment of obesity-related metabolic abnormalities, including dyslipidemia.

One such adipokine is resistin. Circulating resistin levels are increased in obesity and are correlated with body mass index (BMI) and visceral fat content. Resistin is a member of a class of small cysteine-rich secreted signaling proteins, collectively termed resistin-like molecules. In humans, resistin is secreted by both adipocytes and macrophages in adipose tissue. In rodents, there is strong accumulating evidence that increased resistin levels contributes to the pathophysiology of insulin resistance and inflammation. However, studies in humans failed to show a consistent correlation of changes in resistin levels in obesity with these conditions, indicating that rodent data on resistin-induced metabolic changes cannot always be translated to humans.

A recent study investigated gain-of-function effects of resistin on dyslipidemia in mice. The study showed that adenoviral overexpression of murine resistin results in elevated plasma triglycerides and cholesterol and also increased in vivo VLDL-triglyceride production. The quantity of resistin in these mice, however, were well above human physiological levels. Moreover, the effects of resistin on VLDL apoB metabolism, a key determinant of plasma VLDL levels, were not investigated. Finally, effects of resistin on VLDL regulation at the cellular hepatocyte level were not studied. Conversely, another more recent study showed that whole-body gene deletion of resistin in mice that are either genetically obese (ob/ob) or induced to become obese through high-fat feeding results in significant reductions in plasma triglycerides and cholesterol and also reductions in in vivo VLDL-triglyceride secretion. Notably, loss of resistin signaling also markedly reduced hepatic steatosis in the obese mice. Again, similar to the earlier study, the effects of physiological resistin levels were not investigated, neither were VLDL apoB metabolism or VLDL regulation at the hepatocyte level.

Translating the mouse data on resistin's role in dyslipidemia development in obesity to humans is of tremendous potential importance in reducing the alarming ASCVD rates in human obesity. However, species-specific differences in resistin indicate that the results in mice may not necessarily translate to humans. There is only a 53% homology between the human and murine resistin genes. Moreover, some but not all prior studies have found significant associations between circulating resistin levels in humans and VLDL levels. Many of these studies, it should be noted, involved small number of subjects. The more recent studies utilizing more optimized resistin assays do show significant correlations between the two. A recent large population-based Framingham study, for example, did find highly significant associations between serum resistin levels and serum apoB and serum triglycerides. No study, however, has investigated a potential cause and effect relationship between resistin and VLDL levels in humans.

### SUMMARY OF THE INVENTION

It has now been determined that modulation of resistin levels in humans directly effects the in vivo level of lipoproteins.

Accordingly, in one aspect of the invention, a method of modulating plasma levels of lipoproteins in human cells is provided comprising the step of inhibiting the levels of resistin in the cellular environment.

In another aspect of the invention, a method of treating cardiovascular disease in a human is provided comprising the step of inhibiting the expression or activity of resistin in the human.

In a further aspect, a method of screening candidate compounds for inhibition of resistin is provided. The method comprises the steps of:

a) incubating a candidate compound with resistin-expressing sample; and

b) measuring the activity of resistin in the cells, wherein a reduction in the activity of resistin in comparison to a control value obtained in the absence of incubation with the candidate indicates that the candidate compound is a resistin inhibitor.

In another aspect, a method of diagnosing elevated lipoprotein levels in a human subject is provided comprising the step of determining in a resistin-expressing sample obtained from the subject the level or activity of resistin, PCSK9 or MTP, wherein an increase in the level or activity of resistin, PCSK9 or MTP in comparison to a control level is indicative of elevated lipoprotein levels.

These and other aspects of the invention will be described by reference to the detailed description and figures.

### BRIEF DESCRIPTION OF THE FIGURES

FIG. 1 graphically illustrates that human resistin stimulates human hepatocyte apoB 100 protein in a dose-response manner (A) with peak expression observed at 50 ng/mL resistin (B), and that this effect is observed in other species;

FIG. 2 graphically illustrates that the stimulatory effect of human resistin (50 ng/mL) on cellular apoB 100 protein expression and secretion was maintained after 48 hours of treatment (A) and that resistin treatment results in a cumulative effect maximizing at 24 hours (B);

FIG. 3 graphically illustrates that resistin enhanced the stimulatory effect of oleate apoB 100 protein secretion (A), and that resistin does not markedly effect cell viability (B);

FIG. 4 graphically illustrates that resistin treatment of HepG2 cells resulted in an increase in cell apoB mRNA content (A) and that the increase was dose-response with maximal effects observed at 50 ng/mL resistin;

FIG. 5 illustrates the chromatographic analysis of particles secreted as a result of human resistin stimulation of human hepatocytes (A.), and that resistin treatment stimulates the secretion of lipids primarily in the VLDL lipoprotein fraction (B);

FIG. 6 graphically illustrates the relative amounts of lipids expressed in hepatocytes following resistin treatment;

FIG. 7 graphically illustrates the mRNA expression levels of SREBP1 and SREBP2 (A), HMG-coA reductase, HMG-coA synthase and squalene synthase (SS) (B), and the cellular fatty acid (acetyl-coA carboxylase (ACC), fatty acid synthase (FAS), steroyl-coA desaturase (SCD) and triglyceride biosynthesis (DGAT1) as a result of resistin treatment of HepG2 cells;

FIG. 8 graphically illustrates that human resistin stimulates human hepatocyte apoB protein expression and secretion by enhancing intracellular proteosome-mediated apoB stability shown by similar magnitude increases in cellular apoB protein expression of lactacystin and resistin treatment of hepatocytes (A), an increase in human hepatocyte MTP protein expression and activity (B), and decreased human hepatocyte expression of key proteins in the intracellular insulin signaling pathway, including IRS-2, ERK, phosphorylated ERK, Akt and phosphorylated Akt (C);

FIG. 9 graphically illustrates the effect of various concentrations of resistin on hepatocyte LDL receptor protein levels;

FIG. 10 graphically illustrates the effect on hepatocyte LDL receptor (A) and LDL protein (B) levels in cultured hepatocytes in which PCSK9 gene expression was inhibited in the presence and absence of resistin, and LDL receptor (C) and LDL protein (D) levels in hepatocytes isolated from wildtype and PCSK9 knockout mice;

FIG. 11 graphically illustrates the effect of resistin and MTP inhibitor, individually and combined, on SREBP2 mRNA expression and HMG-coA reductase (A), on PCSK9 protein levels (B), and LDL receptor protein levels (C);

FIG. 12 graphically illustrates the effect of antibody removal of resistin in obese and lean human serum on cellular LDL receptor level (A), and PCSK9 expression (B);

FIG. 13 graphically illustrates the effect of lovastatin alone and combined with on hepatocyte LDL receptor expression (A), and on the level of cellular PCSK9 protein (B);

FIG. 14 graphically illustrates the effect of resistin siRNA on cellular levels of resistin, apoB, LDL receptor and PCSK9 levels; and

FIG. 15 illustrates human resistin gene (A) and protein (B) sequences.

### DETAILED DESCRIPTION OF THE INVENTION

A method of modulating the level of lipoprotein in human cells is provided comprising the step of inhibiting the activity of resistin in the cells.

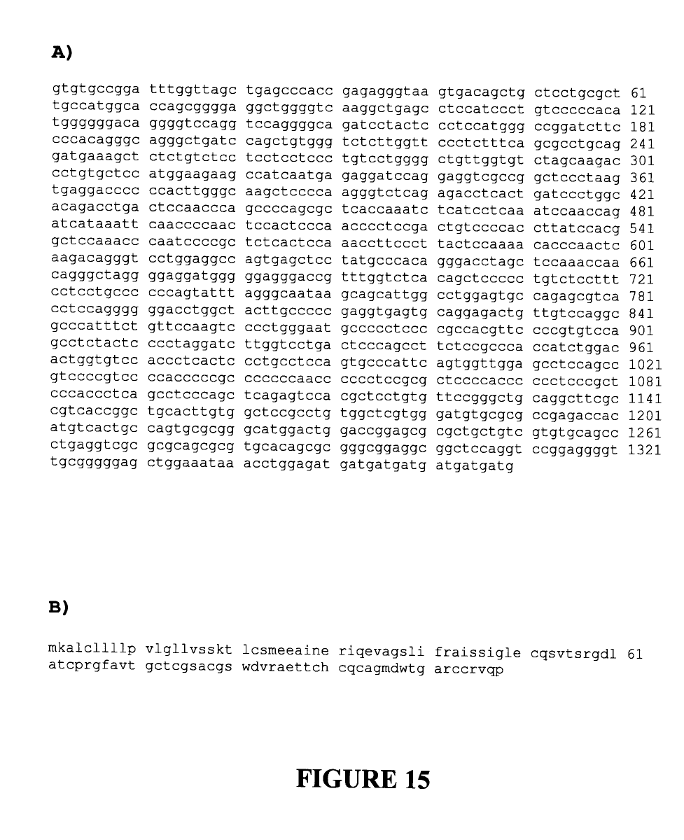

The term “resistin” is used herein to refer to a cysteine-rich cytokine also known as adipose tissue-specific secretory factor (ADSF) or C/EBP-epsilon-regulated myeloid-specific secreted cysteine-rich protein (XCP1). Native human resistin has 108 amino acids, as shown in FIG. 15B, and is encoded by the REIN gene (FIG. 15A). For the purposes of the present invention, the term “resistin” also encompasses functional variants of human resistin. The term “functional variant” refers to a resistin protein that differs from the native protein by one or more amino acid substitutions, additions or deletions, but retains the activity of the native resistin protein, for example, the ability to upregulate cellular lipoproteins, such as very-low-density-lipoprotein (VLDL) and LDL, the ability to upregulate apolipoproteins on lipoproteins, including apolipoprotein B (apoB), and to down-regulate LDL receptors.

In the present method of modulating lipoprotein levels in cells, the activity or expression of resistin may be inhibited within the cellular environment, including intracellularly, extracellularly and within serum. The term “lipoprotein” is used herein to denote undesirable lipoproteins, e.g. lipoproteins associated with an adverse or pathological outcome in a human, including but not limited to, VLDL, LDL, intermediate-density lipoproteins (IDL), apolipoproteins from the lipoproteins such as apoB, apoC and apoE, and lipids from these lipoproteins such as triglycerides (e.g. tracylglycerol), cholesterol and phospholipids. The term “inhibit” as it is used herein with respect to resistin is meant to refer to any reduction of resistin expression or activity including both complete as well as partial reduction of expression or activity. As one of skill in the art will appreciate, inhibition of resistin may be achieved at the nucleic acid level, e.g. inhibition of nucleic levels or expression of the protein, or at the protein level, e.g. inhibition of activity. In either case, the result of inhibiting, or at least reducing, resistin activity is achieved. Inhibition of resistin expression or activity, in accordance with the invention, may be in an amount of at least about 10%, more preferably at least about 20%, 25%, 30%, or greater.

Resistin gene expression may be inhibited using well-established methodologies utilizing polynucleotides, such as anti-sense, snp or siRNA technologies, which are derived from a resistin-encoding nucleic acid molecules such as the sequence shown in FIG. 15A. Such a resistin-encoding nucleic acid sequence, thus, may be used to prepare antisense oligonucleotides effective to bind to resistin nucleic and inhibit the expression thereof. The term “antisense oligonucleotide” as used herein means a nucleotide sequence that is complementary to at least a portion of a target resistin nucleic acid sequence. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides (P. E. Nielson, et al Science 1991, 254, 1497). PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures (U.S. Pat. No. 5,034,506). Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on a given resistin nucleic acid sequence such as that provided herein. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, siRNA technology may be applied to inhibit expression of resistin. Application of nucleic acid fragments such as siRNA fragments that correspond with regions in a resistin gene and which selectively target a resistin gene may be used to block resistin expression. Such blocking occurs when the siRNA fragments bind to the resistin gene thereby preventing translation of the gene to yield functional resistin.

SiRNA, small interfering RNA molecules, corresponding to resistin are made using well-established methods of nucleic acid syntheses as outlined above with respect to antisense oligonucleotides. Since the structure of target resistin genes is known, fragments of RNA that correspond therewith can readily be made. The effectiveness of selected siRNA to block resistin activity can be confirmed using a resistin-expressing cell line. Briefly, selected siRNA may be incubated with a resistin-expressing cell line, such as hepatocytes, under appropriate growth conditions. Following a sufficient reaction time, i.e. for the siRNA to bind with resistin mRNA to result in decreased levels of free resistin mRNA, the reaction mixture is tested to determine if such a decrease has occurred. Suitable siRNA will prevent processing of the resistin gene to yield functional resistin protein. This can be detected by assaying for resistin activity in a cell-based assay, for example, to identify expression of a reporter gene that is regulated by resistin binding, as described in more detail herein.

It will be appreciated by one of skill in the art that siRNA fragments useful in the present method may be derived from specific regions of resistin-encoding nucleic acid which may provide more effective inhibition of gene expression, for example, at the 5′ end or the central region of the gene. In addition, as one of skill in the art will appreciate, useful siRNA fragments need not correspond exactly with a resistin target gene, but may incorporate sequence modifications, for example, addition, deletion or substitution of one or more of the nucleotide bases therein, provided that the modified siRNA retains the ability to bind selectively to the target resistin gene. Selected siRNA fragments may additionally be modified in order to yield fragments that are more desirable for use. For example, siRNA fragments may be modified to attain increased stability in a manner similar to that described for antisense oligonucleotides.

Once prepared, oligonucleotides determined to be useful to inhibit resistin gene expression, such as antisense oligonucleotides and siRNA, may be used in a therapeutic method to modulate, e.g. reduce, the level of lipoproteins in a human subject. A suitable oligonucleotide may be introduced into tissues or cells of the mammal using techniques in the art including vectors (retroviral vectors, adenoviral vectors and DNA virus vectors) or by using physical techniques such as microinjection.

Resistin activity may also be inhibited at the protein level, for example, using inhibitors designed to block resistin either directly or indirectly. Resistin inhibitors may include biological compounds, and synthetic small molecules or peptide mimetics, for example, based on such biological compounds.

Biological resistin inhibitors also include immunological inhibitors such as monoclonal antibodies prepared using well-established hybridoma technology developed by Kohler and Milstein (Nature 256, 495-497 (1975)). Hybridoma cells can be screened immunochemically for production of antibodies specifically reactive with a selected resistin region and the monoclonal antibodies can be isolated. The term “antibody” as used herein is intended to include fragments thereof which also specifically react with a resistin protein according to the invention, as well as chimeric antibody derivatives, i.e., antibody molecules resulting from the combination of a variable non-human animal peptide region and a constant human peptide region.

Candidate resistin inhibitors such as synthetic small molecules or peptide mimetics may also be prepared, for example, based on known biological inhibitors, but which incorporate desirable features such as protease resistance. Generally, such peptide mimetics are designed based on techniques well-established in the art, including computer modelling.

Candidate inhibitors may be screened for inhibitory activity by assaying for resistin activity in a cell-based system. Suitable assays utilize primary or established resistin expressing cell lines, such hepatocyte cell lines. Resistin activity may be monitored in such cell lines by measuring the level of one or more markers of resistin inhibition including, but not limited to, mRNA or protein levels of resistin, LDL receptor level, PCSK9 levels, lipoprotein levels (such as VLDL, LDL and their apolipoprotiens, including apoB, apoC or apoE, and their lipid components) and other outputs such as protein activity, protein modifications, cell function, cell activities, and the like. In the presence of a compound which inhibits resistin, lipoprotein levels will each be reduced in comparison to control levels determined in a resistin-expressing cell line which is incubated in the absence of the candidate compound, while levels of LDL receptor increase in comparison to a control. Lipoprotein levels can be readily detected immunologically, using labelled antibodies directed to apolipoproteins in selected lipoproteins, such as apoB on VLD and LDL, and also by detection of lipids by calorimetry in selected lipoproteins, such as, triglycerides and cholesterol. As will be appreciated by one of skill in the art, the levels of markers of resistin inhibition may also be determined using one or more of a number of standard techniques such as slot blots or western blots (for protein quantitation) or Q-PCR (for mRNA quantitation) in suitable cell culture following incubation with the candidate inhibitor for a suitable period of time, for example 24-48 hours.

A therapeutic inhibitor of resistin may be administered to a human subject to modulate lipoprotein levels in the subject. Inhibitors of resistin expression and inhibitors of resistin activity, including both nucleic acid-based, protein-based and other inhibitors, may be administered in combination with a suitable pharmaceutically acceptable carrier. The expression “pharmaceutically acceptable” means acceptable for use in the pharmaceutical and veterinary arts, i.e. not being unacceptably toxic or otherwise unsuitable. Examples of pharmaceutically acceptable carriers include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the type of inhibitor and the intended mode of administration of the composition. In one embodiment of the invention, the compounds are formulated for administration by infusion, or by injection either subcutaneously, intravenously, intrathecally, intraspinally or as part of an artificial matrix, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

A resistin inhibitor may be administered to a human subject in combination with other therapeutic agents to enhance the treatment protocol. For example, a resistin inhibitor may be co-administered with another drug used to treat elevated serum LDL, including the statins, e.g. lovastatin, atorvastatin, fluvastatin, pitavastatin, pravastatin, rosuvastatin and simvastatin.

The present method of modulating lipoprotein levels may be utilized to treat various pathological conditions in a human subject that result from increased levels of undesirable lipoproteins, e.g. 90 mg/dL or greater of serum apoB, 150 mg/dL or greater of serum triglycerides (derived mostly from VLDL), or 180 mg/dL or greater of serum cholesterol (derived mostly from LDL and VLDL. Such pathological conditions may include, but are not limited to, dyslipidemia, cardiovascular disease such as atherosclerosis, coronary heart disease, myocardial infarction, stroke, venous and arterial thromboembolism, obesity, ischemia, stenosis, angina, diabetes and glucose dysregulation.

To modulate lipoprotein levels in accordance with the present method, a therapeutically effective amount of resistin inhibition is attained by methods such as those described. The term “therapeutically effective” with respect to resistin inhibition is meant to refer to a level of resistin inhibition that reduces undesirable lipoprotein levels to an acceptable level, such as the lipoprotein levels in a healthy control, e.g. a level that is typical of in an individual with a body mass index (BMI) of less than or equal to about 25 kg/m². Acceptable lipoprotein levels may be characterized by a serum apoB level of less than 90 mg/dL, serum triglycerides of less than 150 mg/dL, serum cholesterol of less than 180 mg/dL and LDL-cholesterol of less than 130 mg/dL.

A method of diagnosing elevated lipoprotein levels in a human subject is also provided in another aspect of the invention. The method comprises the step of determining in a resistin-expressing sample obtained from the subject, e.g. plasma, serum, skin fibroblast cells, adipocytes, macrophages and the like, and identifying in the sample one of resistin, PCSK9 or MTP levels or activity using well-established assays such as those described herein. An increase in either of resistin, PCSK9 or MTP levels or activity as compared to a control level, e.g. level in a healthy lean control (having a BMI of less than or equal to about 25 kg/m²), is indicative of elevated lipoprotein levels. Generally, the greater the increase in resistin, PCSK9 or MTP from the control level, the greater the lipoprotein levels in the subject.

Embodiments of the invention are described by reference to the following specific examples which are not to be construed as limiting.

### Example 1

### Methods

Cell Culture.

Cultured Cells: HepG2 cells were obtained from American Type Culture Collection (ATCC, Manassas, Va.). HepG2 cells were grown and maintained in 10% FBS-containing DMEM supplemented with 1% penicillin-streptomycin and 0.06% L-glutamine (584 mg/L) at 37° C., 5% CO₂. During experiments in which HepG2 cells were treated with human recombinant resistin (Calbiochem, UK) the media was changed to 1% FBS-containing DMEM. Cells were stimulated with resistin at various doses (0, 5, 10, 25, 50 and 100 ng/mL) for 24 hours or with 50 ng/mL resistin for various time points (0, 2, 4, 8, 12, 24 and 48 hours). In separate experiments, cells were treated with the fatty acid, oleate (100 μM) (Sigma, ON) for 24 hours, with or without human resistin (50 ng/mL) for 24 hours. In other experiments, cells were treated with the 10 μM lactacystin (Cayman Chemical, Ann Arbor, Mich.) for 24 hours to assess intracellular proteosome-dependent apoB protein degradation.

Cell Culture.

Primary Cells: Fresh wild-type rat and mouse primary heptaocytes were supplied by CellzDirect (Invitrogen, NC) in a 6-well collagen coated plate. Upon arrival, the storage media was removed and incubation media (Williams E Medium, phenol red free, with incubation supplement pack, Gibco, NC) was added according to manufactures' instructions. The cells were incubated at 37° C. with 5% CO₂ for 16 hours prior to human resistin (50 ng/mL) treatment for 24 hours. Cryopreserved plateable human hepatocytes, metabolism qualified from multiple normal human donors, were supplied by CellzDirect (Invitrogen, NC). Upon arrival, the cells (4-8 million in 1 mL), according to the manufacturer's instruction, were added to 48 mL warmed thawing medium (CHRM® Medium, Invitrogen, NC) and centrifuged at 100×g for 10 min at room temperature. The pellet was re-suspended in 4 mL plating medium (Williams E Medium, phenol red free, with maintenance supplement pack and 10% FBS, Gibco, NC). The cells were stained by Trypan Blue (Sigma, Canada) and counted by a haemocytometer, followed by seeding 1×10⁶ cells/well in a 6-well collagen coated plate (CellzDirect, Invitrogen, NC). The cells were incubated at 37° C. with 5% CO₂ for 4 hours to allow the cells to adhere. The plating media was replaced and cell incubated for 16 hours prior to human resistin treatment for 24 hours.

Immunoprecipitation and Western Blots.

Cell lysates collected with RIPA buffer (50 mM Tris, 150 mM sodium chloride, 1% NP-40, 12 mM sodium deoxycholate, 3.5 mM SDS, pH 7.4) and protease inhibitor cocktail (Roche Diagnostics, QC) and media were immunoprecipitated for apoB100, apoCI, apoCIII, apoE, beta-actin or albumin, using the catch-and-release immunoprecipitation columns and kit (Millipore, Billerica, Mass.) for immunocomplex pull-down. Immunoprecipitates containing equivalent amounts of total protein were subjected to sodium dodecyl sulfate polyacrylamide gel electrophoresis (SDS-PAGE), transferred onto nitrocellulose membranes (BioRad, Hercules, Calif.) and immunoblotted for using antibodies as described (Zapata et al. J. Biol. Chem. 1998, 273:12, 6916-6920) against the following proteins: apoB100 (human and rodent) (Santa Cruz, Calif.), apoCI (Santa Cruz, Calif.), apoCIII (Santa Cruz, Calif.), apoE (Santa Cruz, Calif.), beta-actin (Sigma-Aldrich, St. Louis, Mo.), albumin (Santa Cruz, Calif.), IRS-2 (Millipore, CA), extracellular signal-related kinase (ERK) and phosphorylated ERK (Cell Signalling Technology, MA), Akt and serine and threonine phosphorylated Akt (Cell Signalling Technology, MA). Horseradish peroxidase-conjugated antibodies (BioRad, Hercules, Calif.) were used as secondary antibodies. Immunoreactive bands were visualized with a chemiluminescence kit (PerkinElmer Life Sciences, Waltham, Mass.). The blots were exposed to KODAK Biomax films, and the signal was quantified by densitometry using Quantity One version 4.6.7. software (Bio Rad, Hercules, Calif.).

Real-Time Quantitative PCR Analysis.

Total RNA was isolated from cell lysates (RNeasy Mini Kit, Qiagen, Germantown, Md.) and used as a template for cDNA synthesis (QuantiTech Reverse Transcription Kit, Qiagen, Germantown, Md.). Quantitative real-time PCR was performed using an Applied Biosystems 7300 Real Time PCR system (Carlsbad, Calif.) according to the manufacturer's instructions and with the SYBR green master kit (Qiagen, Germantown, Md.). Primers for the real-time PCR internal control gene cyclophilin A (sense, 5′-GTCAACCCCACCGTGTTCTTC-3′ (SEQ ID NO:3); antisense, 5′-TTTCTGCTGTCTTTGGGACCTTG-3′ (SEQ ID NO:4)) were synthesized (IDT, Coralville, Iowa). Primers for succinate degydrogenase (SDH) (another real-time PCR internal control gene), apoB, microsomal triglyceride transfer protein (MTP), Srebp1, Srebp2, acetyl-coA carboxylase (ACC), HMG-coA reductase, HMG-coA synthase, squalene synthase (SS), the LDL receptor, PCSK9, fatty acid synthase (FAS), sterol desaturase (SCD), DGAT1 and DGAT2 were purchased (proprietary sequences not available) (Qiagen, Germantown, Md.). The values reported for each mRNA were corrected to the cyclophilin A and SDH mRNA values.

Oil-Red-0 Staining.

Cells were stained with Oil-Red-0 to examine the amount of neutral lipid accumulation in the cells as described by Ferre et al. (Am. J. Physiol Gastrointest. Liver Physiol. 296:G553-G562). Briefly, dishes were washed with cold phosphate-buffered saline and fixed in 10% neutral formalin. After 2 changes of propylene glycol, Oil-Red-0 was added with agitation for 7 minutes, followed by washing in 85% propylene glycol. The dishes were then rinsed in distilled water and counterstained with hematoxylin. For each dish, 3 images were photographed, and a representative image is shown.

Electron Microscopy.

The VLDL lipoprotein fraction was isolated from HepG2 cell media via ultracentrifugation at density of 1.006 g/mL using a Beckman Optima TL ultracentrifuge and 100.4 TLA rotor (Beckman Coulter, Brea, Calif.). Mean particle size for VLDL was then determined using the Hitachi 7000 electron microscope equipped with a AMT XR-60 digital camera. The fixation process for electron microscopy utilized 1% Os04 in a phosphate buffer at pH 7.4 applied to the VLDL fraction for a 30 minute exposure. A small drop of this solution was placed on a 400 mesh copper grid coated with a carbon film and allowed to stand for 3 minutes or until sample has dried. The fixed VLDL were placed on the electron microscope grid for viewing and digitizing. The captured images were taken at 75 KV using a beam current of 25 uA. Digital Images of the VDL were taken at about 50,000×. The mean particle size was then determined by importing the digital images into NIH image J software.

Lipid Measurement.

Lipids from HepG2 cell extracts were quantified by gas chromatography (GC) as described in Sahoo et al. (J. Lipid Res. 45:1122-1131). Briefly, cell extracts were incubated with phospholipase C (Sigma, ON) to remove polar head groups, then extracted in the presence of internal standard by the method of Folch et al. (J Biol Chem 226:497-509). Extracted lipids were passed through a sodium sulfate column to remove aqueous contaminants, and derivatized with Sylon BFT (Supelco, ON) to cap reactive hydroxyl and carboxyl groups. Derivatized lipids were dissolved in hexane and injected onto a Zebron ZB-5 column (Phenomenex, Torrance, Calif.) in an Agilent 6890 GC instrument. Lipoprotein Profiles. HepG2 media from 6-10 cm plates was collected and concentrated using an Amicon Centriprep concentrator 50 K and lipoprotein classes separated by fast protein liquid chromatography (FPLC) on a Superose 6 10/30 gel filtration column (Amersham, UK) followed by inline post-column reaction with either Infinity Triglyceride or Infinity Cholesterol reagent (Thermo Scientific, West Palm Beach, Fla.) and measurement of absorbance @ 500 nm.

Cell Viability.

Cell viability was determined with 0.4% tryphan blue (Sigma-Aldrich, ON) staining and calculated using the following formula: % Cell Viability=(number of unstained (living) cells/Total number of cells)×100.

Microsomal Triglyceride Transfer Protein (MTP) Activity Assay.

Cell monolayers were washed twice with ice-cold PBS and once with 5 ml of 1 mM Tris-HCl, pH 7.6, 1 mM EGTA, and 1 mM MgCl₂ at 4° C. Cells were then incubated for 2 min at room temperature in 5 ml of ice-cold 1 mM Tris-HCl, pH 7.6, 1 mM EGTA, and 1 mM MgCl₂. The buffer was aspirated, and 0.5 ml of the same buffer was added to cells. Cells were scraped and collected in ice-cold tubes, vortexed, and centrifuged (SW55 Ti rotor, 50,000 rpm, 10° C., 1 h), and supernatants were used for MTP assay using the MTP fluorometric activity assay kit (Chylos Inc., NY). The triglyceride transfer activity of MTP is presented as % transfer/h/mg protein.

Statistical Analysis.

Statistically analyzed data were analyzed using t-tests or ANOVAs, depending on the experimental conditions. All results are presented as means±SEM. Unless otherwise indicated, asterisks (*) and (**) and crosses (†) indicate statistically significant differences (P<0.05, P<0.01 and P<0.001, respectively) compared with respective controls.

### Results

Human Resistin Directly Stimulates Apolipoprotein (Apo) B Expression and Secretion in Human Hepatic HepG2 Cells.

Cultured human hepatic HepG2 cells were maintained in 10% FBS-DMEM until confluent in 10 cm plates. Once 80-90% confluent, HepG2 cells in 1% FBS-DMEM were treated with recombinant lyophilized purified human resistin (Calbiochem) reconstituted in millipore H₂0. It was confirmed that the human resistin source was indeed purified human resistin by applying the reconstituted human resistin to a 10% denaturing SDS-PAGE gel and using a rabbit polyclonal resistin antibody against human resistin (Santa Cruz) for detection. A single 12 kDa band was observed in the resulting immunoblot.

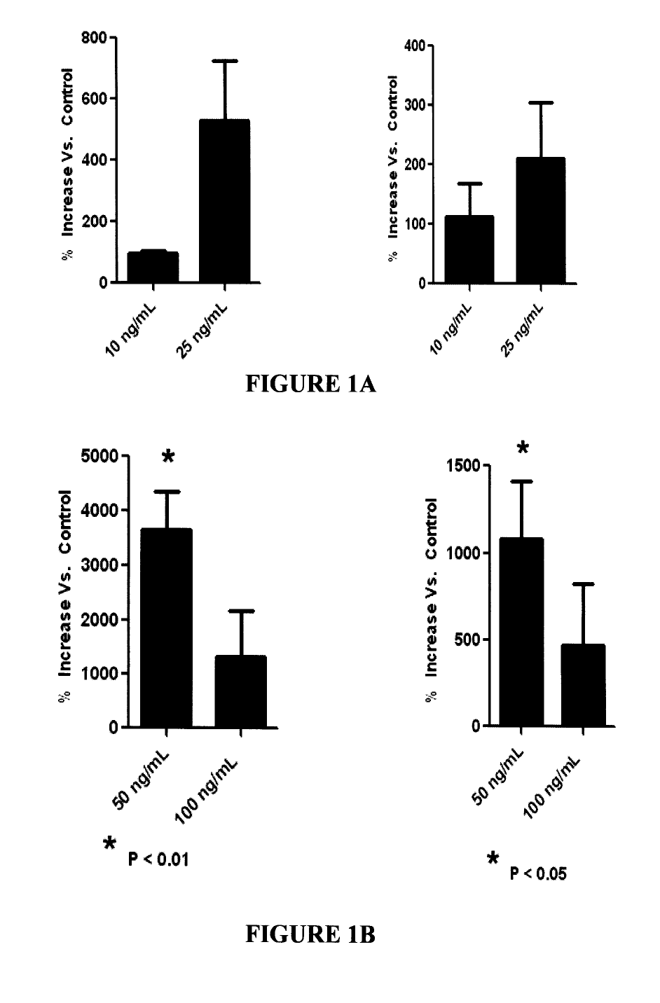

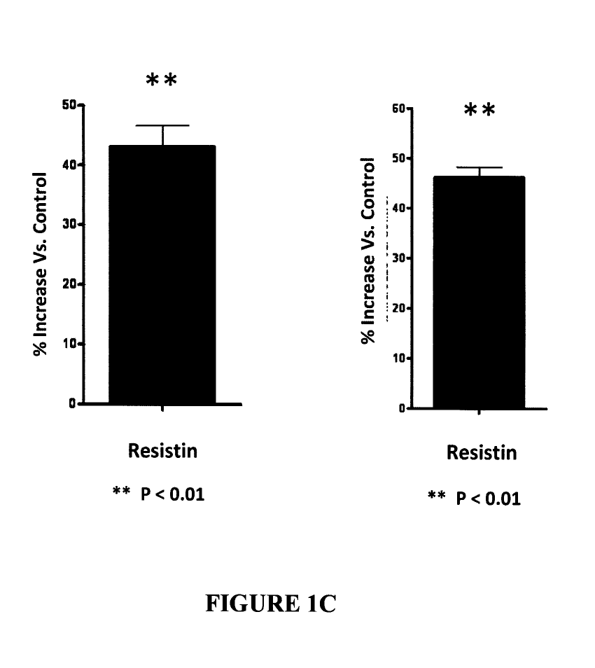

All of the experiments to follow were performed three times and representative results are shown. Dose-response experiments in HepG2 cells, treating the cells with 0, 10, 25, 50 and 100 ng/mL of human resistin for 24 hours were performed. Immunoprecipitation on cell lysates and media, followed by Western blots to detect the cellular expression and secretion of human apoB protein were then performed. The constitutively expressed cellular proteins, beta-actin and albumin were used as internal immunoblot controls to confirm equal protein loading in the apoB immunoblots of cell lysates and media, respectively. Treatment with resistin at the concentration typically found in lean humans—10 ng/mL—resulted in a marked doubling (a nearly 100% increase) of hepatic apoB cellular protein expression and secretion versus control, untreated HepG2 cells (FIG. 1A). Treatment with 25 ng/mL resistin further stimulated apoB secretion by approximately 250% compared with untreated cells (FIG. 1A).

Remarkably, addition of 50 ng/mL human resistin to HepG2 cells resulted in an approximately 3500% increase in cellular apoB protein expression and a 1000% increase in secreted apoB versus untreated cells (FIG. 1B). This finding demonstrates the highly potent effect of human resistin in mediating the production of apoB-containing lipoprotein particles and indicates a highly pro-atherogenic role for human resistin not previously identified.

Because the cellular expression of apoB protein was much greater than apoB secretion into the media with 50 ng/mL resistin, this indicates that much of the apoB synthesized as a result of resistin treatment is degraded before secretion. Nonetheless, the 1000% elevation of apoB secretion induced by 50 ng/mL resistin treatment above control, untreated cellular levels, would be expected to greatly deteriorate the dyslipidemic profile of obese individuals since the plasma levels of apoB-containing lipoproteins in humans are largely determined by their hepatic rates of production.

While a clear dose-response positive relationship was seen between 0-50 ng/mL human resisin on HepG2 apoB protein secretion, treatment of HepG2 cells with 100 ng/mL resistin, surprisingly reduced cellular apoB expression markedly and reduced its secretion versus that produced by 50 ng/mL resistin (FIG. 1B). This amount of resistin—100 ng/mL—is a supraphysiological amount of resistin that produced a negative feedback effect on apoB protein production and/or stability.

Human Resistin Directly Stimulates Apolipoprotein (Apo) B Expression in Primary Human and Rodent Hepatic Cells.

To determine if the stimulatory effect of human resistin on hepatocyte apoB protein expression is relevant in vivo, freshly isolated mouse and rat hepatocytes and cryogenically preserved isolated human hepatocytes were treated with human resistin at the optimum 50 ng/mL dose for 24 hours. Note that the recommended media for the primary hepatocytes was not optimal for maintaining the stability of apoB protein secreted into the media and thus robust apoB protein bands were not identified from media. However, cellular apoB 100 protein expression was clearly visible from primary cell lysates. As with the HepG2 cell results above, significantly greater apoB protein expression was observed in human resistin treated cells versus untreated control cells in all species tested (mouse, rat and human) (FIG. 1C). The percentage increases seen were in the 40-50% range for all three species confirming that the resistin stimulatory effect was applicable in vivo. While the magnitude of the stimulatory effect of human resistin on cellular apoB protein expression in primary hepatocytes was not as large as with HepG2 cells, this was expected as primary hepatocytes are not as metabolically active as cultured hepatocytes.

Human Resistin Stimulation of Hepatic ApoB Expression and Secretion is Prolonged, Rapid, More Potent than Oleate and does not Induce Cellular Apoptosis in HepG2 Cells.

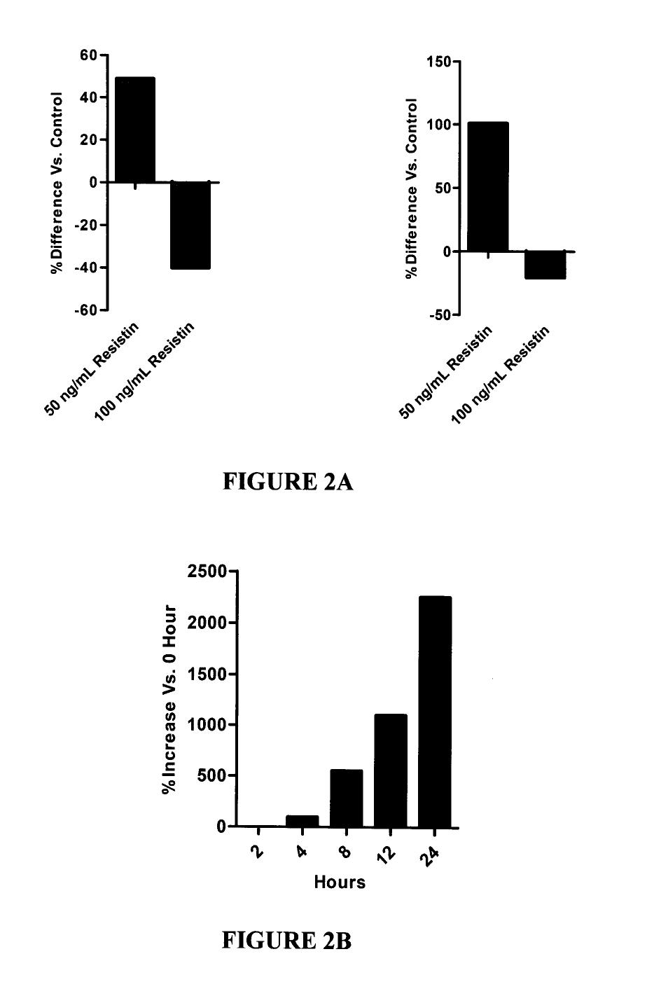

To determine whether the acute stimulatory effect of human resistin on hepatic apoB protein expression and secretion is maintained for a prolonged period of time, the following was conducted. The stimulatory effect of 50 ng/mL resistin treatment (the dose of resistin found to be most deleterious on hepatocyte apoB secretion) on HepG2 apoB secretion observed at 24 hours was maintained after 48 hours, albeit at a lower magnitude (100% above untreated HepG2 cells) (FIG. 2A.). HepG2 cells were not restimulated with resistin after the initial addition of resistin to HepG2 cell media at the start of the experiment. This observation on the prolonged stimulatory effect of human resistin on hepatic apoB can be interpreted in at least two ways. One interpretation is that the resistin peptide is very stable in cellular media; another possibility is that resistin induces a prolonged enhancement of the cellular machinery that stimulates apoB production and/or stability.

The time-course effects of human resistin on hepatic apoB secretion was then studied. A steady increase in apoB protein secretion into HepG2 cell media was observed as early as 4 hours after resistin treatment and up until the 24-hour length of the experiment, when the maximal cumulative effect on apoB protein expression in the media was observed (FIG. 2B.). Since apoB protein synthesis in human hepatocytes has been reported to require 8 hours, this indicates that cellular mechanisms which are more rapid than stimulation of apoB protein translation are enhanced by human resistin. Enhanced apoB stability by resistin is therefore a likely mechanism by which human resistin increases cellular apoB protein. Stimulation of hepatic cellular enzymes which enhance apoB stability (particularly microsomal triglyceride transfer protein (MTP), decreased expression of key proteins in the intracellular insulin signaling pathway, or increased cellular neutral lipid content, all of which are known to enhance apoB stability, are plausible mechanisms.

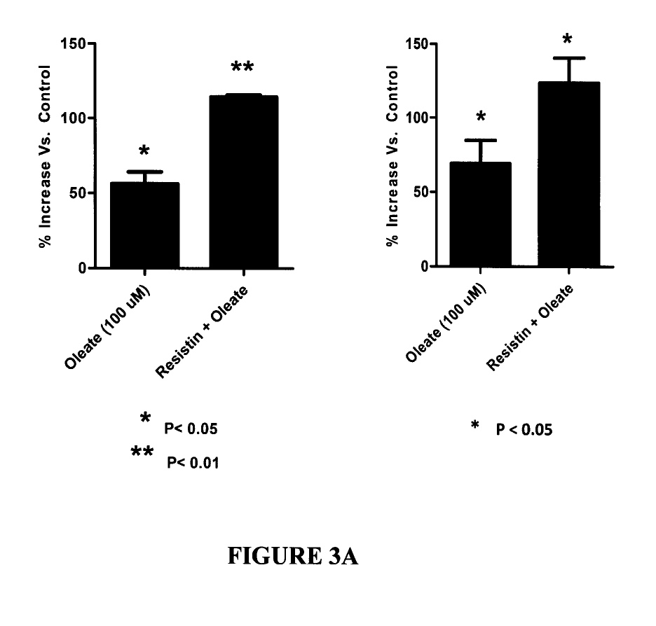

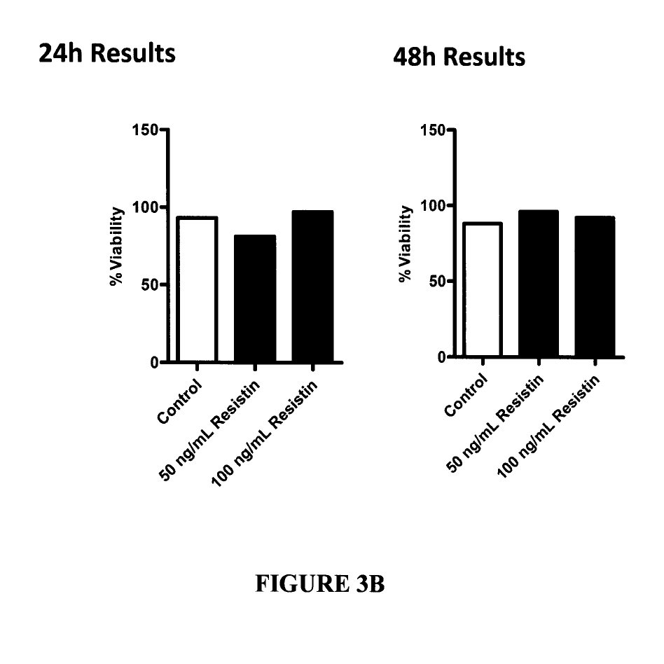

To determine the magnitude or potency of the effect of human resistin on hepatic apoB expression and secretion, the effect of the abundant plasma monounsaturated free fatty acid (FFA), oleate, traditionally and commonly used to stimulate hepatic apoB production, was compared to that of resistin. The addition of 50 ng/mL of resistin to 100 μM of oleate for 24 hours doubled the stimulatory effect of the oleate on hepatic cellular apoB expression and secretion (FIG. 3A). To give a clearer idea of the potency of human resistin to oleate, 50 ng/mL of resistin is equivalent to 4.6 pM or 4.6×10⁻¹² μM of resistin, which is comparative to 100 μM of oleate. Thus, human resistin is a highly potent therapeutic human drug target.

The next question was whether this amount of human resistin is toxic to hepatocytes. Tryphan blue staining of HepG2 cells upon 50 ng/mL of resistin treatment to determine cell viability was performed. The results showed no marked reduction in hepatocyte viability with either 50 ng/mL or 100 ng/mL human resistin treatment for 24 or 48 hours (FIG. 3B.).

Human Resistin Stimulation of ApoB is Partly Mediated at the mRNA Level.

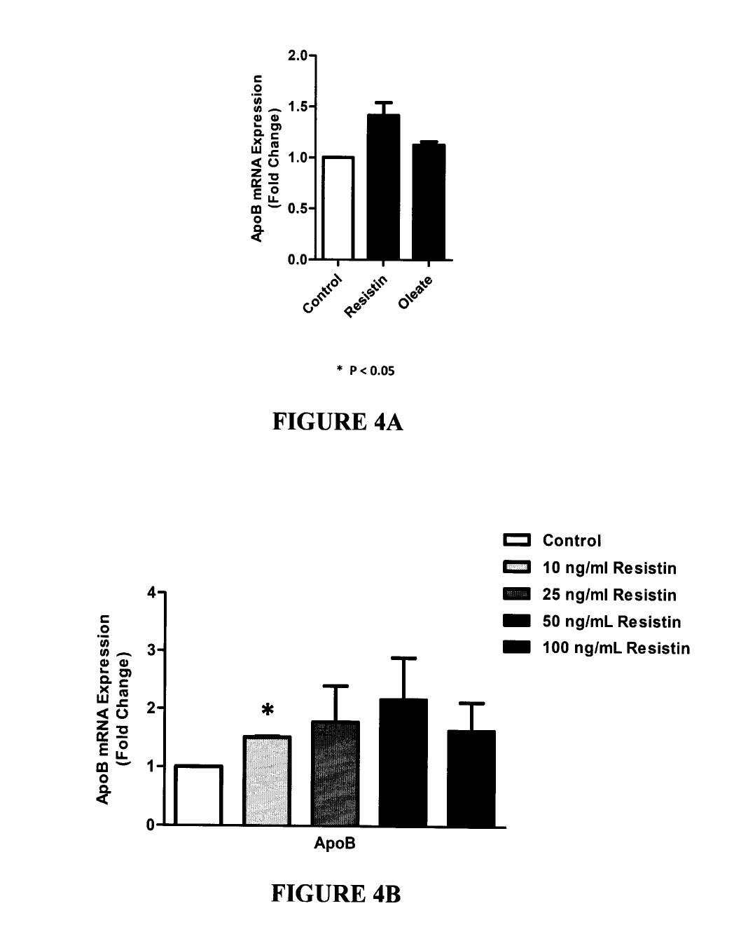

Results from real-time RT-PCR analyses demonstrated that human resistin stimulation of hepatocyte apoB expression is partly mediated at the transcriptional level. There was a significant increase in apoB mRNA expression in HepG2 cells with 50 ng/mL human resistin treatment for 24 hours (FIG. 4A). ApoB mRNA expression increased in a dose-response fashion from 0, 10, 25 to 50 ng/mL human resistin treatment and then decreased at a 100 ng/mL resistin dose, paralleling the pattern seen with hepatocyte apoB protein expression with human resistin stimulation (FIG. 4B).

### Human Resistin Stimulates the Secretion of Atherogenic Very-Low-Density Lipoprotein (VLDL) Particles in HepG2 Cells.

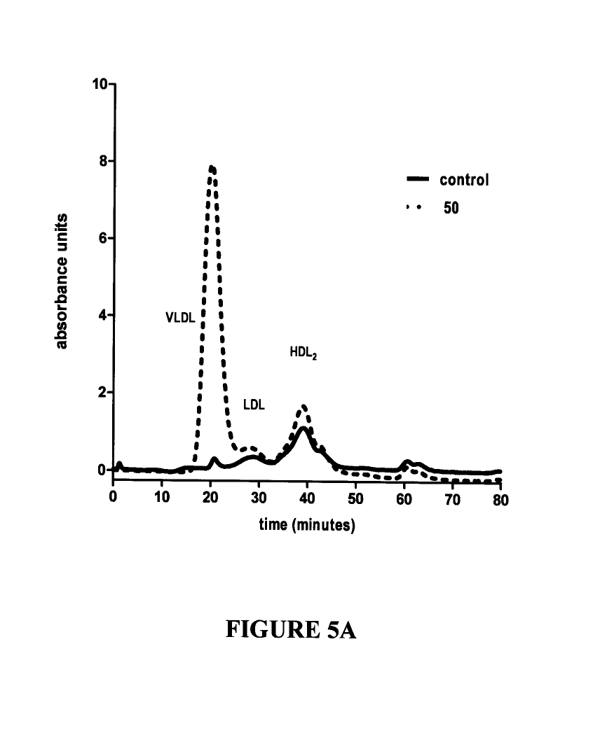

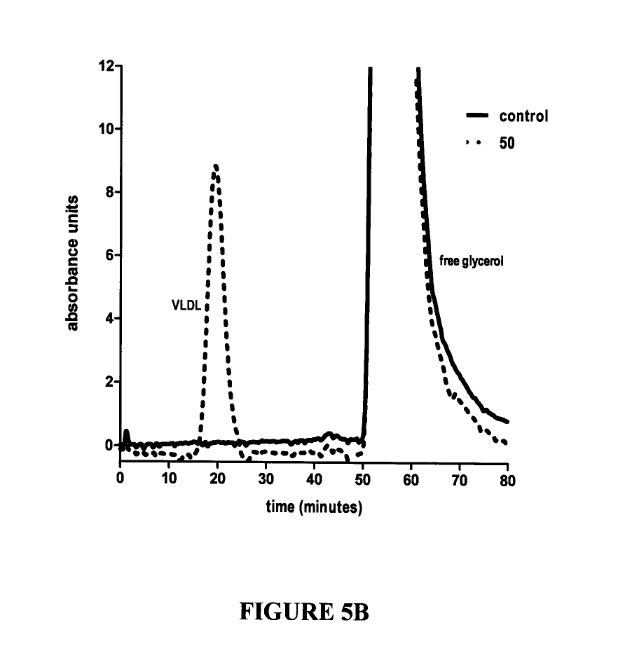

The characteristics of the apoB-containing lipoprotein particles produced as a result of treatment of human hepatocytes with human resistin was determined. Fast protein liquid chromatography (FPLC) (size exclusion chromatography) was used to determine the effect of human resistin treatment on the lipid composition of lipoproteins secreted by HepG2 cells. Thus, after 24 hour incubation of HepG2 cells with human resistin at a concentration of 50 ng/mL, the most effective resistin dose, and including an untreated control HepG2 sample, the HepG2 cell media from 6-10 cm plates was collected and concentrated from each treatment group. The concentrated media was then injected into Superdex FPLC columns, fractionated and eluted and individual fractions were analyzed for triglycerides and cholesterol. The results showed large increases in the triglyceride and cholesterol contents of the secreted VLDL fraction as a result of human resistin treatment, with virtually no change in lipid contents of the secreted LDL or HDL fractions. The magnitude of the increase in secreted VLDL triglycerides and cholesterol was over 8-9-fold with human resistin treatment, relative to control untreated cells (FIGS. 5A and 5B.).

The secretion of apoB protein by HepG2 cells increased markedly by approximately 8-fold with human resistin treatment, along with the observed increases in secreted VLDL lipids. Thus, increases in secreted VLDL lipid components by HepG2 cells treated with resistin should be a reflection of increased secreted VLDL particle numbers compared to control, untreated hepatocytes, with each individual VLDL particle having less triglyceride and cholesterol contents to that of secreted VLDL particles by control, untreated HepG2 cells. This would indicate that the type of VLDL particles secreted by resistin-treated hepatocytes are smaller and denser and therefore more atherogenic than that secreted by control, untreated hepatocytes. To confirm whether this is indeed the case, electron microscopy (EM) analyses was performed on media from HepG2 cells either untreated or treated with 50 ng/mL for 24 hours. The VLDL fraction from the media was first isolated via density ultracentrifugation of the media at d1.006 g/mL. The VLDL fraction was then fixed and stained with osmium and imaged using EM. The EM analyses using the NIH Image J software program showed a mean VLDL diameter of 80 nm in the resistin-treated samples, which was less than that of control untreated sample (e.g. 110 nm). Furthermore, the quantity of VLDL particles found in a representative 100 cm² area was increased markedly in resistin-treated samples by a mean of 10-fold.

The apoC and apoE protein expression by HepG2 cells treated with human resistin (50 ng/mL) was characterized and compared to that secreted by control, untreated HepG2 cells. There was no difference in apoCI or CIII protein expression in HepG2 cells treated with or without resistin. This indicates that the VLDL particles secreted by hepatocytes treated with human resistin are no different in terms of their capacity to activate lipoprotein lipase mediated lipolysis, a major circulatory remodeling enzyme. There was, however, an increase in the expression of secreted apoE in human resistin-treated hepatocytes, which is a ligand for hepatic lipase, which does induce lipoprotein lipolysis at hepatocytes and can induce the formation of smaller, denser lipoprotein particles.

### Human Resistin Induces Hepatic Neutral Lipid Accumulation in HepG2 Cells.

To determine if the deleterious effects of human resistin extend not only to increased secretion of apoB-containing lipoproteins by human hepatocytes, but also to increased hepatocyte lipid content, as the two processes frequently occur together in such conditions as obesity, insulin resistance and type 2 diabetes mellitus. Oil-Red-O/hematoxylin staining of HepG2 cells either untreated, treated with 100 μM oleate for 24 hours as a positive control, or treated with 50 ng/mL human resistin for 24 hours was performed. The results showed a clear increase in hepatocyte neutral lipid content in human resistin-treated HepG2 cells versus control, untreated HepG2 cells. The increased cell neutral lipid content with resistin treatment was similar in magnitude to that observed with 100 μM oleate treatment.

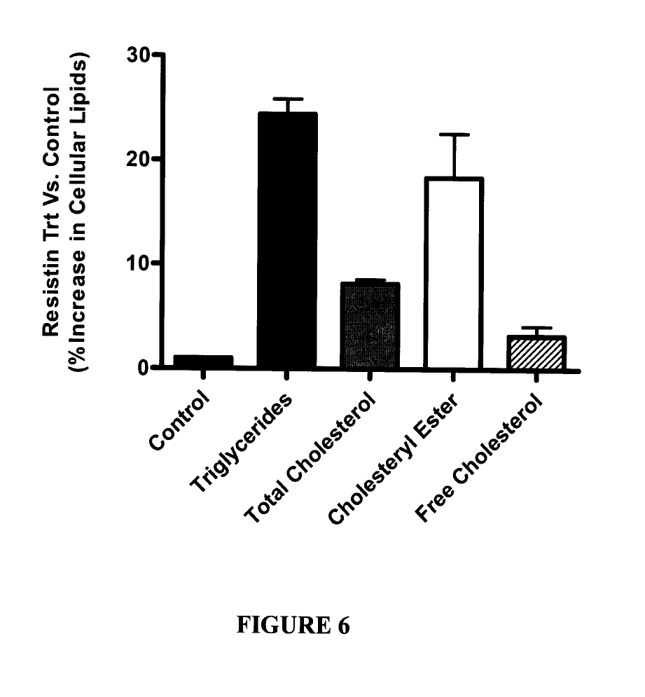

To quantify the hepatocyte lipid changes induced by human resistin, gas chromatography (GC) analyses of lipids extracted from harvested hepatocytes either untreated or treated with human resistin (50 ng/mL) for 24 hours was performed. GC analyses showed a 24% increase in triglyceride content, a 18% increase in cholesteryl ester content, and a 3% increase in the free cholesterol content in hepatocytes treated with resistin for 24 hours (FIG. 6). Therefore, the results indicate that human resistin acts acutely to markedly increase hepatocyte triglycerides and cholesterol and can potentially directly induce fatty liver and hepatic steatosis concomitant with increasing hepatic VLDL secretion.

### Human Resistin Mediated Increased Cellular Neutral Lipid Content and VLDL Lipid Secretion is Via Induction of the Cellular SREBP1 and SREBP2 Lipogenic Pathways.

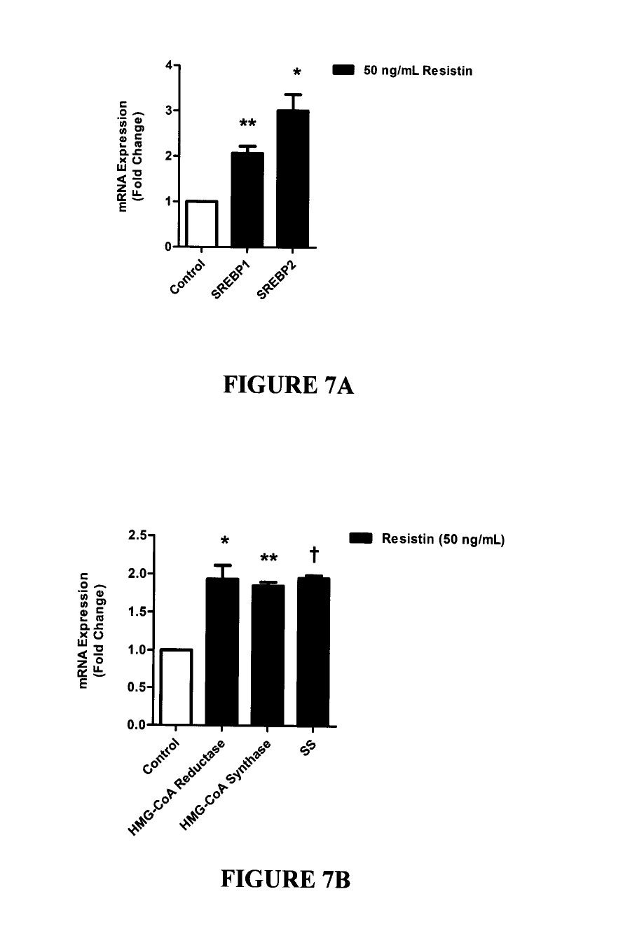

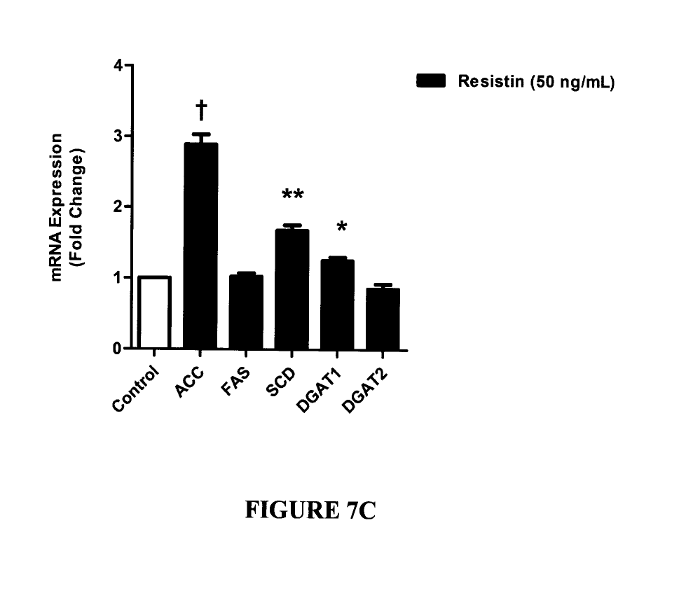

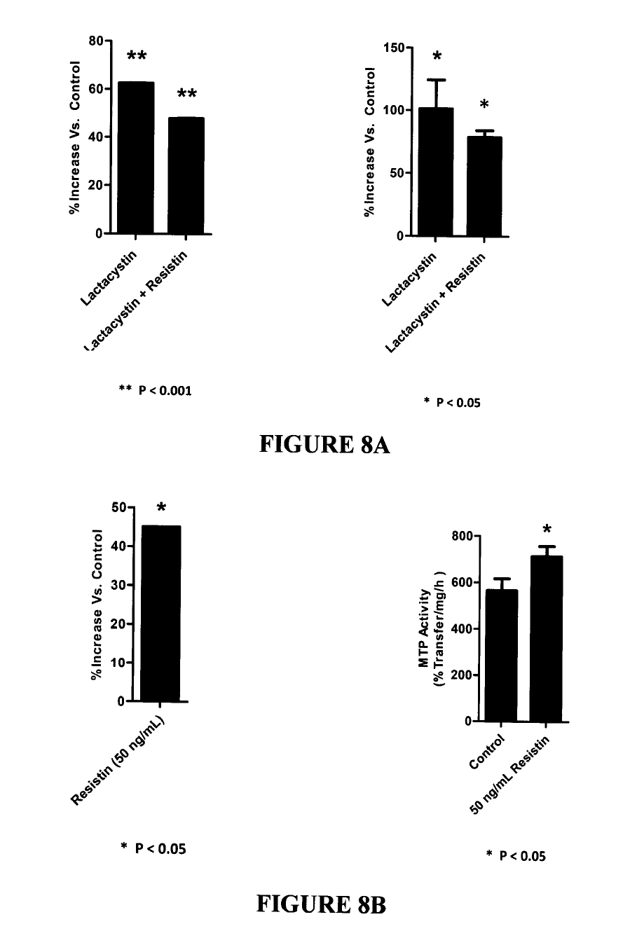

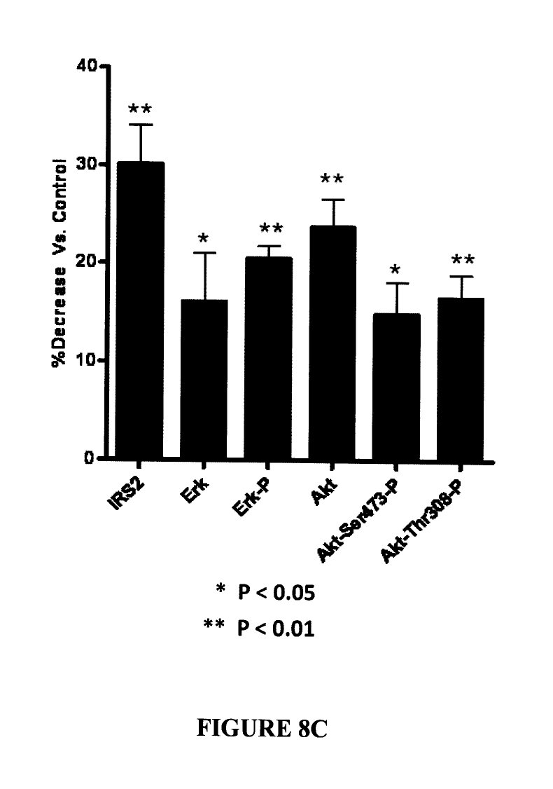

Tests were then conducted to determine whether the increase in resistin-treated hepatocyte and secreted VLDL neutral lipid content is due to increased cellular de novo lipogenesis. Since elevated intracellular neutral lipids enhance intracellular apoB protein stability, this explains in part the enhanced apoB protein expression and subsequent increase in apoB protein secretion observed with hepatocyte human resistin treatment. Indeed, the results showed significantly increased mRNA expression of SREBP1 and SREBP2 genes, the master transcription factors in the fatty acid/triglyceride and cholesterol cellular biosynthesis pathways, respectively, upon hepatocyte human resistin treatment (50 ng/mL, 24 hours) (FIG. 7A.). This was associated with a significant 3-fold increase in the expression of ACC and significant 2-fold increases in the expression of key SREBP2 intracellular cholesterol biosynthetic target genes: HMG-CoA REDUCTASE, HMG-CoA SYNTHASE and SQUALENE SYNTHASE (FIG. 7B.). The expression of genes involved in cellular cholesterol uptake—the LDL RECEPTOR and PCSK9—also increased (FIG. 8C.). A decline in LDL receptors induced by resistin would be expected to further increase circulating VLDL levels in humans beyond that induced by the resistin-mediated increase in VLDL secretion and due to decreased hepatocyte VLDL uptake. In terms of the SREBP1 pathway, SCD, which mediates intracellular monounsaturated fatty acid biosynthesis, and DGAT1, which mediates intracellular triglyceride biosynthesis, increased to smaller but significant extents with hepatocyte human resistin treatment (FIG. 7C.).

Key Mechanisms by which Human Resistin Mediated Increased Hepatocyte ApoB Expression and Secretion is Mediated is Via Increased Microsomal Triglyceride Transfer Protein (MTP) Activity and Reduced Insulin Signaling.

Further tests were conducted to investigate mechanisms by which human resistin stimulated increased hepatocyte expression and secretion of apoB protein. Since the time-course hepatocyte apoB secretion study described above shows a much more rapid increase in apoB secretion than would be required for increased apoB transcription, and because it is well known that apoB is primarily regulated by co- and post-translational mechanisms—particularly co- and post-translational apoB degradation, regulators of posttranslational cellular apoB degradation were investigated. It was first determined whether human resistin inhibits apoB degradation through the classic proteosome-dependent degradation pathway by adding the optimal dose of human resistin (50 ng/mL) to the proteosome-dependent inhibitor of apoB degradation, lactacystin, at the optimal dose and time (10 μM, 24 hours). An increase in the cellular expression and secretion of apoB in HepG2 cells with lactacystin was observed, confirming its apoB degradation properties (FIG. 8A.). The addition of 50 ng/mL human resistin to lactacystin, however, did not further increase apoB expression or secretion, indicating that human resistin acts to increase hepatocyte apoB primarily through inhibition of proteosome-mediated apoB degradation. Increased hepatocyte availability of lipids for incorporation with apoB is a key mechanism by which apoB degradation is inhibited and indeed, as indicated in the section above, human resistin increased cellular neutral lipid content that can then have been accessed by apoB during its assembly into VLDL particles intracellularly. The intracellular enzyme, MTP, is crucial for the transfer of such lipids to apoB and is thereby a key regulator of intracellular apoB stability. Assessment of cellular MTP protein expression and, more importantly, activity, in HepG2 cells in response to human resistin treatment (50 ng/mL, 24 hours) showed significant increases in both MTP parameters, demonstrating for the first time, that human resistin directly stimulates MTP in hepatocytes (FIG. 8B.).

Another important regulator of hepatocyte apoB/VLDL assembly and stability is the intracellular insulin signaling pathway. Reduced signaling activity in this pathway has been shown to enhance apoB stability both directly and in part by enhancing the cellular availability of lipid and increased MTP expression. It was found that human resistin (50 ng/mL, 24 hours) did indeed significantly decrease the expression of key proteins in the insulin signaling pathway—IRS-2, ERK, phosphorylated ERK, Akt and serine and threonine phosphorylated Akt—by approximately 20-30% (FIG. 8C).

### Discussion

In conclusion, it is shown here for the first time in humans that resistin stimulates hepatocyte oversecretion of VLDL apoB and lipids. At Physiological concentrations, human resistin acts directly and potently on its own in a dose-responsive manner, and also potentiates the stimulatory effects of oleate, in hepatocyte apoB secretion. In particular, human resistin stimulates the production of increased numbers of smaller, more atherogenic VLDL particles. Concomitantly, human resistin markedly increased neutral lipid content of hepatocytes, potentially also causing fatty liver and hepatic steatosis in humans. In view of these results, human resistin is therefore a therapeutic target to ameliorate the epidemic of dyslipidemia and ASCVD in humans.

### Example 2

### HepG2 Cell Culture

HepG2 cells were obtained from American Type Culture Collection (ATCC, Manassas, Va.). Cells were propagated in 10% FBS-containing DMEM supplemented with 1% penicillin-streptomycin and 0.06% L-glutamine (584 mg/L) at 37° C., 5% CO₂. During treatment experiments with human recombinant resistin (Calbiochem, UK) and/or lovastatin (Sigma, Mo.), the media was changed to 1% FBS-containing DMEM. All of the experiments to follow were performed at least three times and representative results are shown. Cells were stimulated with resistin at various doses (0, 10, 25, 50, and 100 ng/mL) for 24 hours or with a single dose at 50 ng/mL resistin. In other experiments, the microsomal triglyceride transfer protein (MTP) inhibitor, CP-346086, was administered at 1.3 nM, either alone or together with resistin (50 ng/mL) for 24 hours. Lovastatin was administered at 1 to 5 μM for 24 hours to stimulate the cells, either alone or in combination with resistin (50 ng/mL). Unless otherwise indicated, all experiments were performed in triplicate in three independent experiments.

Primary Human Hepatocyte Cell Culture.

Cryopreserved plateable human hepatocytes, metabolism qualified from multiple normal human donors, were obtained from CellzDirect (Invitrogen, NC). The cells were seeded at 1×10⁶ cells/well in a 6-well collagen coated plate according to the manufacturer's instructions (CellzDirect, Invitrogen, NC). The cells were incubated at 37° C. with 5% CO₂ for 4 hours to allow the cells to adhere. The plating media was replaced and the cells incubated for 16 hours prior to human resistin 50 ng/mL treatment for 24 hours.

Immunoblotting for Resistin, LDL Receptor and PCSK9.

Recombinant purified human resistin source (Calbiochem, UK) was confirmed via denaturing SDS-PAGE (10%) using a rabbit polyclonal antibody against human resistin (Santa Cruz) for detection. Cell lysates collected with RIPA buffer (50 mM Tris, 150 mM sodium chloride, 1% NP-40, 12 mM sodium deoxycholate, 3.5 mM SDS, pH 7.4) and protease inhibitor cocktail (Roche Diagnostics, QC) were analyzed for human LDL receptor and PCSK9 as previously described (Rashid et al. Proc Nall Acad Sci U S A 2005; 102:5374-9). Equivalent amounts of total protein, determined by Bradford reaction was subjected to sodium dodecyl sulfate polyacrylamide gel electrophoresis (SDS-PAGE), transferred onto nitrocellulose membranes (BioRad, CA) and immunoblotted using antibodies against the LDL receptor (Fitzgerald International, CA) and PCSK9 (Cayman Chemicals, MI). Horseradish peroxidase-conjugated antibodies (BioRad, CA) were used as secondary antibodies. Immunoreactive bands were visualized with a chemiluminescence kit (PerkinElmer Life Sciences, MA). The blots were exposed to KODAK Biomax films, and the signal was quantified by densitometry using Quantity One version 4.6.7. software (Bio Rad, CA).

Real-Time Quantitative PCR Analysis.

Total RNA was isolated from cell lysates (RNeasy Mini Kit, Qiagen, MD) and used as a template for cDNA synthesis (QuantiTech Reverse Transcription Kit, Qiagen, MD). Quantitative real-time PCR was performed using an Applied Biosystems 7300 Real-Time PCR system (Carlsbad, Calif.) according to the manufacturer's instructions and with the SYBR green kit (Qiagen, MD). Primers for the real-time PCR internal control gene cyclophilin A, as identified in Example 1, were synthesized (IDT, IA). Primers for succinate dehydrogenase (SDH) (another real-time PCR internal control gene), SREBP2, HMG-CoA reductase, the LDL receptor and PCSK9 were purchased (proprietary sequences not available) (Qiagen, MD). The values reported for each mRNA were corrected to the cyclophilin A and SDH mRNA values.

siRNA Studies.

Four siRNAs targeting human PCSK9 synthesized by Qiagen (Qiagen, MD) were purchased and tested. The siRNAs were transfected into HepG2 cells at a final concentration of 10 nM, using HiPerFect transfection reagent (Qiagen, MD) at 0.5% final volume. The siRNA that produced the maximum decrease in PCSK9 mRNA and protein expression in HepG2 cells (70% knockdown by Western blot 24 hours post-transfection) was chosen for further experiments. As a negative control, HepG2 cells were transfected with a scrambled siRNA control vector in HiPerFect (both Qiagen, MD). As a positive control, HepG2 cells were transfected with siRNA targeting the constitutively expressed GAPDH gene, using HiPerFect (Qiagen, MD). Western blots against PCSK9 (Caymen Chemicals, MI), LDL receptor (Fitzgerald International, CA) and GAPDH (Sigma, Mo.) were done in control untreated HepG2 cells, cells transfected with the optimal PCSK9 siRNA (Qiagen, MD) and cells treated with human resistin (50 ng/mL) plus PCSK9 siRNA 24 hours post-transfection.

Primary Mouse Hepatocyte Studies.

Animal ethics approval was received for the mouse protocols to follow. Primary mouse hepatocytes from PCSK9 knockout mice (C57Bl/6 background) or their wildtype littermates were isolated by perfusing the liver via the hepatic portal vein with a 5 mM EGTA in Leffert's Buffer (10 mM Hepes; 3 mM KCl, 0.13 M NaCl, 1 mM NaH₂PO₄, 10 mM D-glucose) followed by a 0.3 mg/ml collagenase in 0.0279% CaCl₂. Cells were then filtered through a 70 uM nylon strainer and centrifuged at 760 rpm for 5 min. The cells were then passed through a CHRM gradient (Invitrogen, NC). Viability of cells ranged from 80 to 95%. Mouse hepatocytes were seeded into rat tail Collagen I coated 96-well plates at 30,000 cells/well in complete DMEM incubation media (10% FBS, 1 mM NaPyruvate, 100 nM insulin, 100 nM Dexamethasone) and incubated at 37° C., 5% CO₂ overnight prior to treatment with human recombinant resistin (50 ng/mL) (Peprotech, QC) for 24 hours in complete DMEM incubation media. Mouse LDL receptor and PCSK9 protein in cell lysates were measured via immunoblotting, as described above, using the following primary antibodies: mouse LDL receptor (abcam, MA), mouse PCSK9 (abcam, MA) and mouse GAPDH (Cell Signaling, MA). Horseradish peroxidase-conjugated antibodies (BioRad, Hercules, Calif. and Santa Cruz Biotechnology, CA) were used as secondary antibodies.

Immunoprecipitation-Antibody Removal of Resistin from Human Serum.

Serum was obtained from metabolically well-characterized healthy lean (body mass index (BMI)≦25 kg/m² and waist circumference <102 cm) and obese males (body mass index (BMI)>30 kg/m² and <35 kg/m² and waist circumference >102 cm). All participants provided informed consent and human ethics approval was received for the human serum protocols. Serum resistin concentrations in study participants were measured via ELISA (R&D Systems, Minneapolis, Minn.). To determine the effect of resistin antibody removal on human serum stimulation of cellular LDL receptor and PCSK9 in human hepatocytes, human resistin was immunoprecipitated from serum using the Catch-and-Release immunoprecipitation columns and kit (Millipore, MA). After equilibration of the columns with PBS, human serum was incubated with the beads in the column, along with resistin antibody (Santa Cruz, Calif.), PBS buffer and affinity ligand (supplied in kit), with end over end rotation at 4° C. for 90 minutes. Concentrations were according to the manufacture's instructions. As a control, serum was also incubated with PBS buffer without resistin antibody or affinity ligand. The columns were centrifugated at 2000×g for 5 minutes and the flow-through was used in treatment of human hepatocytes for 24 hours. The columns were thereafter washed and eluted to confirm that resistin was captured when the resistin antibody was included in the immunoprecipitation incubations. After 24 hours of hepatocyte treatment with the resistin immunoprecipitated serum, human LDL receptor and PCSK9 protein in cell lysates were measured via immunoblotting, as described above.

Statistical Analysis.

Statistically analyzed data were analyzed using t-tests or one-way ANOVA, depending on the experimental conditions. All results are presented as means±SEM. Unless otherwise indicated, asterisks ((*) and (**)) indicate statistically significant differences (P<0.05 and P<0.01, respectively) compared with respective controls.

### Results

Cultured human hepatic HepG2 cells (ATCC) were maintained in 1% FBS-DMEM and treated with recombinant purified human resistin (Calbiochem), confirmed via denaturing SDS-PAGE (10%) using a rabbit polyclonal antibody against human resistin (Santa Cruz) for detection. A single 12 kDa band was observed in the resulting immunoblot, confirming that the resistin source was indeed purified human resistin.

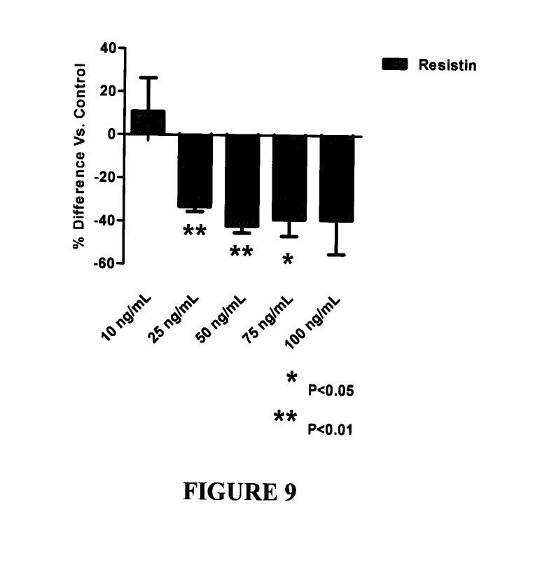

All of the experiments were performed at least three times and representative results are shown. Dose-response experiments were performed in HepG2 cells with 0, 10, 25, 50, 75 and 100 ng/mL of human resistin for 24 hours. Western blots were then performed on cell lysates to detect the cellular expression of LDL receptor protein. Beta-actin was used as an internal control to confirm equal protein loading. As shown in FIG. 9, treatment with resistin at 10 ng/mL, a concentration of resistin characteristic of normal lean humans, did not result in a significant change in hepatocyte LDL receptor protein compared with untreated control cells. In contrast, treatment with 25 ng/mL resistin, an upper level of resistin reported in lean humans, reduced LDL receptor protein expression by 30%. Addition of 50 or 75 ng/mL resistin to HepG2 cells, concentrations of resistin in the range typically reported for obese individuals, resulted in further substantial 40% decreases in cellular LDL receptor protein expression. Such reductions in hepatocyte LDL receptors would be expected to greatly deteriorate the dyslipidemic profile of obese individuals. Furthermore, treatment with 100 ng/mL of resistin, a supraphysiological quantity of resistin, resulted in a similar 40% reduction in hepatocyte LDL receptor expression, indicating the nadir of LDL receptor decline with human resistin treatment.

To determine if the above findings in immortalized HepG2 cells are relevant to humans, primary human hepatocytes isolated from fresh human livers (Invitrogen) were treated with human resistin at the optimum 50 ng/mL dose for 24 hours. As with the HepG2 cell results above, significantly reduced LDL receptor protein in resistin treated cells versus untreated control cells. While the magnitude of the inhibitory effect of human resistin on LDL receptor protein expression was not as large as with HepG2 cells, this was expected as primary hepatocytes are not as metabolically active as cultured hepatoma HepG2 hepatocytes.

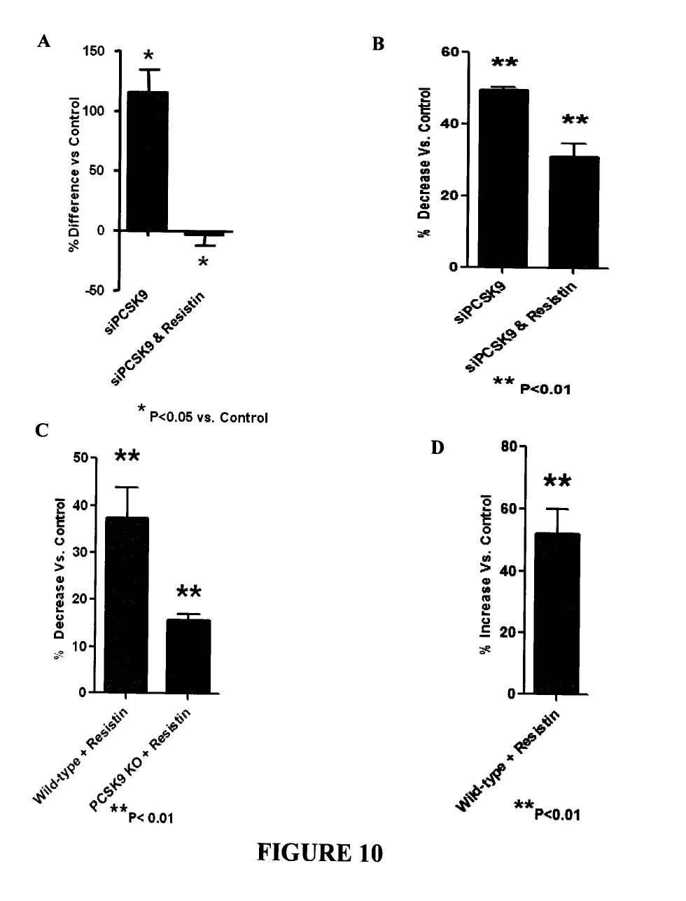

The extent of the role of PCSK9 in the resistin mediated reduction in hepatocyte LDL receptor levels was examined. PCSK9 gene expression was inhibited in hepatocytes via PCSK9 siRNA treatment for 24 hours, which inhibited PCSK9 mRNA levels significantly by 60%, compared to vehicle control hepatocytes incubated with transfection reagent alone. The addition of resistin reversed the marked over 100% elevation in hepatocyte LDL receptor expression induced with PCSK9 siRNA treatment (FIG. 10A). PCSK9 protein levels in hepatocytes were next assessed in response to PCSK9 siRNA administration, with and without resistin. The results showed that the addition of resistin with PCSK9 siRNA enhanced cellular PCSK9 levels, compared with siRNA treatment alone (FIG. 10B). This indicates that resistin had stabilized hepatocyte PCSK9 protein. Overall, these findings indicate that the reduction in cellular LDL receptor protein levels by resistin and enhanced LDL receptor degradation occurs, at least in part, via upregulation of PCSK9. This is a novel function of human resistin and is the first identification of a natural serum factor directly regulating PCSK9 protein levels in hepatocytes.

To then more directly quantify the role of PCSK9 in the resistin mediated reduction in hepatocyte LDL receptor levels, hepatocytes isolated and cultured from wild-type mice were treated with resistin, and compared to resistin-treated hepatocytes from PCSK9 knockout mice. As expected, in wild-type mice hepatocytes, resistin markedly decreased LDL receptor protein levels, by 40% (FIG. 10C), compared with untreated hepatocytes, and also increased PCSK9 levels significantly (FIG. 10D), similar to the findings in human hepatocytes. Resistin also significantly decreased LDL receptor expression in hepatocytes from PCSK9 knockout mice, but the magnitude of the effect was reduced to a 15% decline in LDL receptor expression compared to untreated hepatocytes from PCSK9 knockout mice (FIG. 10D). These findings show that the elevation in PCSK9 protein levels induced by resistin plays a major, but not exclusive, role in the reduction of cellular LDL receptor levels mediated by resistin.

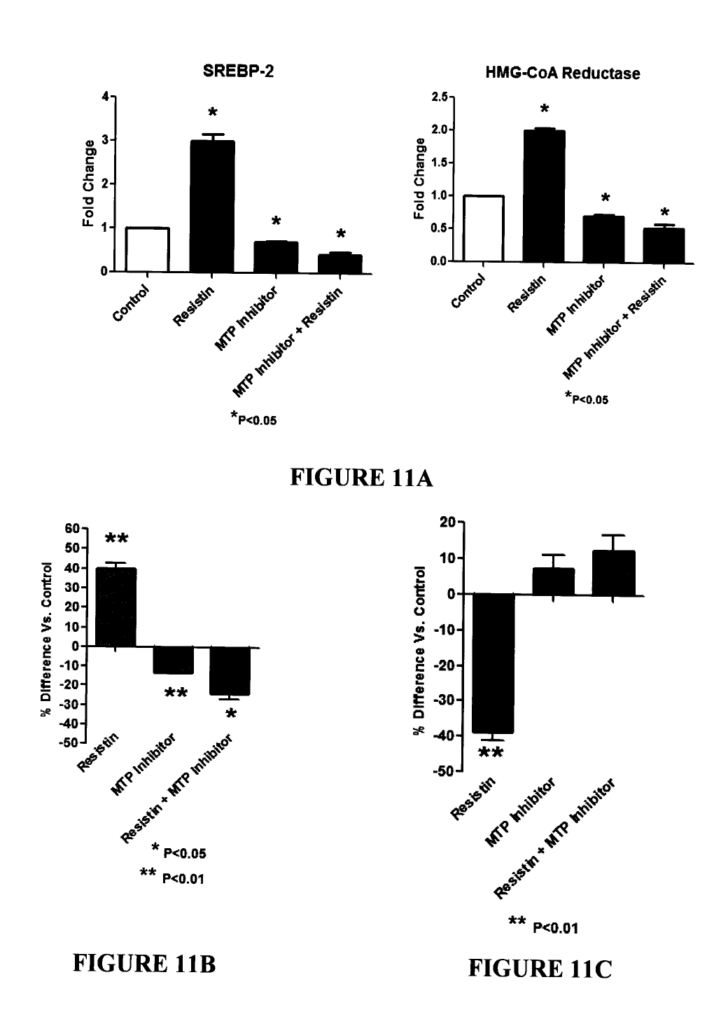

As shown in Example 1, resistin stimulates hepatocyte synthesis and secretion of very-low-density lipoproteins (VLDL), an effect that is mediated by increased activity of the rate-limiting intracellular protein in VLDL production, microsomal triglyceride transfer protein (MTP). MTP accelerates the transfer of neutral lipids, including cholesteryl esters, to apolipoprotein B intracellularly, for their eventual egress from cells. Conversely, MTP inhibitors, as a class, reduce this egress of lipids from the cell, thereby causing cellular accumulation of lipids. It was then determined if excess accumulation of intracellular lipids through MTP inhibition would ameliorate the cellular upregulation of SREBP2, and its targets, particularly PCSK9, induced by resistin, thereby ameliorating the effect of resistin. This was another method by which the role of SREBP2 and PCSK9 in the resistin mediated reduction in hepatocyte LDL receptors could be quantitifed. MTP inhibition, via CP-346086, at a non-toxic dose that did not reduce cell viability (1.3 nM), did induce a significant reduction in cellular de novo cholesterol synthesis, as shown by a reduction in SREBP2 mRNA expression and its target gene, HMG-coA reductase, rate-limiting in cellular cholesterol biosynthesis (FIG. 11A.). This reduction in SREBP2 by CP-346086, thereby, reversed the increase in PCSK9 protein levels induced by resistin (FIG. 11B), and, thus, reversed the decline in LDL receptor protein levels observed with resistin treatment (FIG. 11C). These results indicate that the SREBP2-mediated elevation in hepatocyte PCSK9 levels is necessary for the decline in cellular LDL receptor levels induced by resistin.

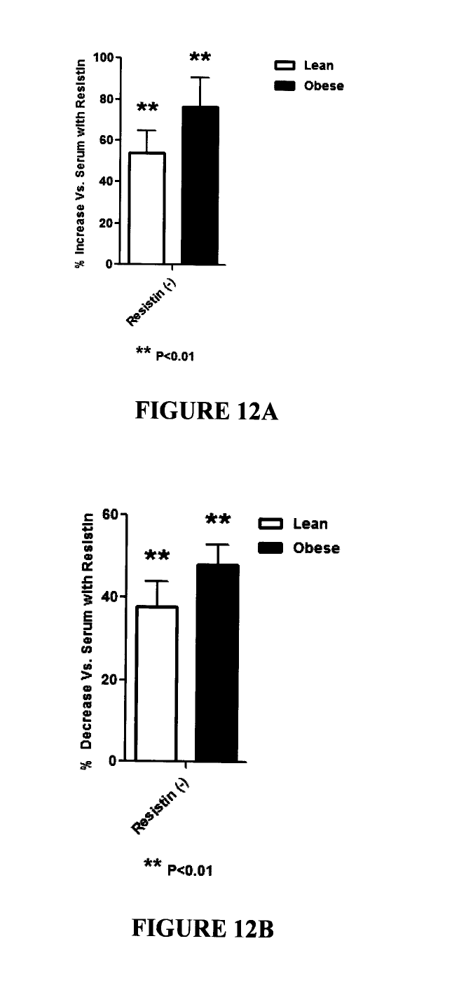

Immunoprecipitation-antibody removal of resistin from human serum was performed, and the subsequent effect on hepatocyte LDL receptor expression was examined. Antibody removal of resistin in obese human serum reversed the obese serum mediated reduction in cellular LDL receptors remarkably by 80% (FIG. 12A), an effect that was mediated at least in part by reduced PCSK9 expression (by 50%) (FIG. 12B). Removal of resistin in lean human serum also increased hepatocyte LDL receptor levels, albeit to a lesser extent than resistin removal from obese human serum (FIG. 12A). These results indicate that resistin in human serum plays a quantitatively important role in mediating hepatocyte LDL receptor levels. This further indicates that reduction or inhibition of serum resistin in humans is a potentially effective treatment for elevated LDL, particularly in obese states.

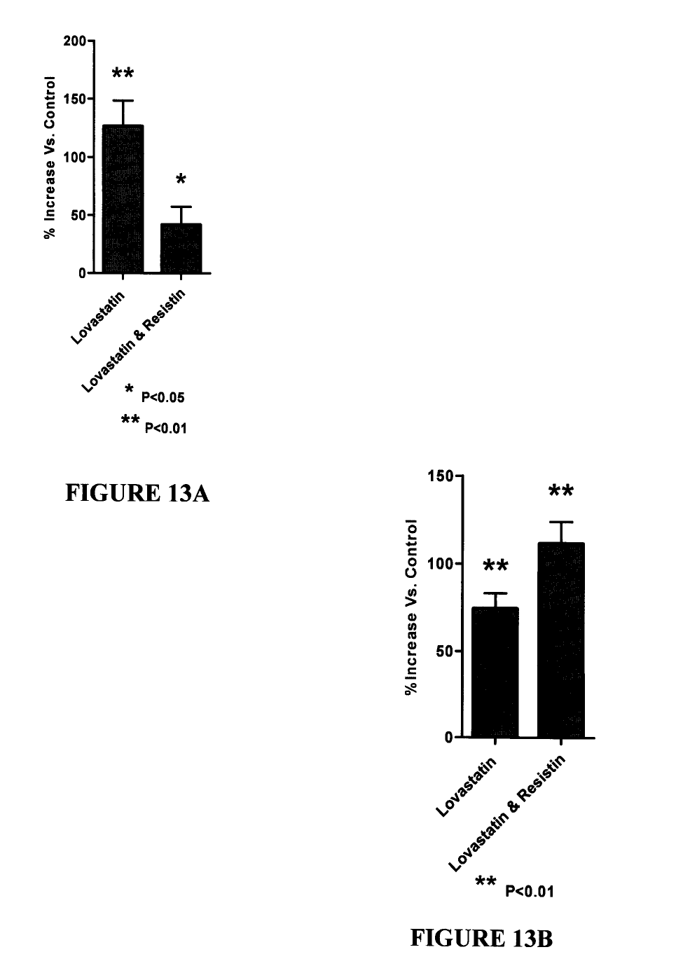

The major class of drugs currently administered to patients with elevated serum LDL are statins. Statins function by reducing cellular cholesterol levels, which activates SREBP2, leading to the transcriptional activation of the LDL receptor. Since resistin stimulates PCSK9, it was determined whether or not the resistin mediated increase in PCSK9 expression, which should be more prevalent in obese individuals, attenuates the increase in LDL receptor expression in patients administered statins. Resistin (50 ng/mL, 24 hours) was found to diminish the increase in hepatocyte LDL receptor expression induced by lovastatin treatment (5 μM) considerably by 70% (FIG. 13A). This inhibition of statin induced LDL receptor upregulation by resistin was attributed at least in part to increased cellular PCSK9 protein (by 50%) (FIG. 13B). These results indicate that inhibitors of resistin can enhance statin-induced hepatocyte LDL receptor expression and reduce plasma LDL levels.

It was then determined if, in a physiologically relevant human setting, resistin inhibits hepatocyte LDL receptor protein expression. HepG2 cells were incubated with serum (10% in DMEM for 24 hours) from healthy obese males (BMI >30 kg/m² and <35 kg/m² and waist circumference >102 cm) with high resistin concentrations (40% elevated compared with lean subjects) and compared them to HepG2 cells incubated with serum from lean males (BMI ≦25 kg/m² and waist circumference <102 cm). The results demonstrated a significant 30% inhibitory effect of obese human serum on cellular LDL receptor protein expression versus lean serum incubation of hepatocytes, and a 40% elevation in PCSK9 levels with obese versus lean serum incubation of hepatocytes.

### Example 3

### Inhibition of Resistin Hepatic ApoB Secretion

### Methods

Cell Culture.

Cultured Hepatoma Cells: HepG2 cells were obtained from American Type Culture Collection (ATCC, Manassas, Va.). HepG2 cells were grown and maintained in 10% FBS-containing DMEM supplemented with 1% penicillin-streptomycin and 0.06% L-glutamine (584 mg/L) at 37° C., 5% CO2. During experiments in which HepG2 cells were treated with human recombinant resistin (Calbiochem, UK), the media was changed to 1% FBS-containing DMEM. Unless otherwise indicated, all experiments were performed in triplicate as three independent experiments. During HepG2 incubations with human serum, the media was replaced with DMEM plus 10% human serum from either lean (body mass index (BMI) ≦25 kg/m2) or obese (BMI >30 kg/m2 and <35 kg/m2) male Multicultural Community Health Assessment Trial (M-CHAT) study participants (described below) for 24 hours.

Immunoprecipitation and Western Blots.

Cell lysates, collected with RIPA buffer (50 mM Tris, 150 mM sodium chloride, 1% NP-40, 12 mM sodium deoxycholate, 3.5 mM SDS, pH 7.4) and protease inhibitor cocktail (Roche Diagnostics, QC), and media were immunoprecipitated for apoB 100, apoCI, apoCIII, apoE, beta-actin, albumin, AMP-activated protein kinase (AMPK), phosphorylated AMP kinase (pAMPK(Thr 172)), acetyl-CoA carboxylase (ACC), insulin receptor substrate (IRS)-2, extracellular signal-related kinase (ERK), phosphorylated ERK (pERK or p44/42 MAPK, phosphorylated on p44 residues Thr202/Tyr204 and p42 residues Thr185/Tyr187), Akt and serine and threonine phosphorylated Akt ((pAkt(Ser473)) and pAkt(Thr(308))) using Catch-and-Release immunoprecipitation columns and kits (Millipore, Billerica, Mass.) for immunocomplex pull-down. Immunoprecipitates containing equivalent amounts of total protein were subjected to sodium dodecyl sulfate polyacrylamide gel electrophoresis (SDS-PAGE), transferred onto nitrocellulose membranes (BioRad, Hercules, Calif.) and immunoblotted using antibodies4 against the following proteins: apoB 100 (human and rodent) (Santa Cruz, Calif.), apoCI (Santa Cruz, Calif.), apoCIII (Santa Cruz, Calif.), apoE (Santa Cruz, Calif.), beta-actin (Sigma-Aldrich, St. Louis, Mo.), albumin (Santa Cruz, Calif.), IRS-2 (Millipore, CA), ERK and pERK (Cell Signaling Technology, MA), Akt, pAkt(Ser473) and pAkt(Thr308) (Cell Signaling Technology, MA) and AMPK and pAMPK(Thr172) (both antibodies were generous gifts generated in-house from Dr. Gregory Steinberg, McMaster University). Horseradish peroxidase-conjugated antibodies (BioRad, Hercules, Calif.) were used as secondary antibodies. Immunoreactive bands were visualized with a chemiluminescence kit (PerkinElmer Life Sciences, Waltham, Mass.). The blots were exposed to KODAK Biomax films, and the signal was quantified by densitometry using Quantity One version 4.6.7. software (Bio Rad, Hercules, Calif.).

Real-Time Quantitative Reverse-Transcriptase (RT)-PCR Analysis. This was conducted as described above.

Lipid Measurements.

Lipids from cell extracts obtained from 3-10 cm plates were pooled and quantified three times by gas chromatography (GC), as previously described (Sahoo et al.).

Human Subjects.

Serum from participants recruited for the Multicultural Community Health Assessment Trial (M-CHAT) (Lear et al. Am J Clin Nutr 2000:86:353-359) was used in the present investigation for stimulation of cellular and secreted apoB protein in human hepatocytes. The M-CHAT study consisted of a multiethnic cohort of healthy men and women, matched for ethnicity and BMI, between 30 and 65 years of age. Those who had a recent weight change (±2.2 kg in 3 months), had a previous diagnosis of CVD or significant comorbidity (such as HIV, an immunocompromised condition, type 1 diabetes mellitus), or had significant prosthetics or amputations were excluded. Those who were currently taking medications for CVD risk factors (i.e. lipid lowering, antihypertensive, or hypoglycemic medications) were also excluded. All participants provided informed consent. This study was approved by the Simon Fraser University Research Ethics Board. BMI was calculated as weight in kilograms divided by height in meters squared. Waist circumference (WC) was the average of 2 measurements taken against the skin at the point of maximal narrowing of the waist. Fasting blood samples were collected and immediately processed for total cholesterol, HDL cholesterol (HDL-C), triglycerides and glucose. All measurements were carried out in the same clinical laboratory with standard enzymatic procedures. For the present study, human serum was obtained from a multiethnic cohort (European and South Asian descent) of 36 exclusively male M-CHAT study subjects.

Resistin ELISA.

The Quantikine Human Resistin ELISA kit was purchased from R&D Systems (Minneapolis, Minn.) to measure serum resistin concentrations in M-CHAT study participants from whom serum was used in hepatocyte apoB stimulation experiments. Serum resistin measurements were performed according to manufacturer's instructions. In brief, serum was diluted 5-fold in the diluent supplied and incubated with the buffer supplied for 2 hours at room temperature in a 96 well plate. The plate was washed and resistin conjugate was added to each well for 2 hours. Following a second wash, substrate solution was added for 30 minutes and the reaction was completed by addition of a stop solution. The plate was read at 450 nm with a correction set at 570 nm. All samples were measured in duplicate.

Resistin Immunoprecipitation.

To determine the effect of resistin antibody removal on human serum stimulation of cellular and secreted apoB in human hepatocytes, human resistin was immunoprecipitated from serum using Catch-and-Release immunoprecipitation columns and kits (Millipore, Billerica, Mass.). After equilibration of the columns with PBS, human serum was incubated with the beads in the column, along with resistin antibody (Santa Cruz, Calif.), PBS buffer and affinity ligand (supplied in the kits), with end over end rotation at 4° C. for 90 minutes, according to the manufacturer's instructions. As a control, serum was also incubated with PBS buffer without resistin antibody or affinity ligand. The columns were centrifugated at 2000 g for 5 minutes and the flow-through was used for treatment of human hepatocytes for 24 hours. The columns were, thereafter, washed and eluted to confirm that resistin was captured when the resistin antibody was included in the immunoprecipitation incubations. After 24 hours of hepatocyte treatment with the resistin-immunoprecipitated serum, apoB protein in cell and media were measured via immunoprecipitation and Western blotting, as described above.

Statistical Analysis.

Data were statistically analyzed using t-tests or one-way ANOVA, depending on the experimental conditions. All results are presented as mean±SEM. Unless otherwise indicated, asterisks ((*) and (**)) indicate statistically significant differences (P<0.05 and P<0.01, respectively) compared with respective controls.

### Results

HepG2 cells with serum (10% in DMEM for 24 hours) from metabolically well characterized obese (19 individuals with BMI >30 kg/m2 and <35 kg/m2) and lean (17 individuals with BMI < or =25 kg/m2) humans from the multiethnic M-CHAT (Multicultural Community Health Assessment Trial) study. For the present study, serum was obtained solely from male patients from the M-CHAT study (N=36). Patients used in the present study had a mean age of 50 years. Lean individuals had a mean BMI of 23 kg/m2, a mean waist circumference (WC) of 84 cm, whereas obese individuals had a mean BMI of 32 kg/m2 and a mean waist circumference of 106 cm. Subjects from both European white and South Asian ancestry were included. Lean and obese subjects had similar serum total cholesterol and LDL-cholesterol levels and similar glucose levels. As expected, obese subjects had significantly greater serum triglyceride and lower HDL-cholesterol concentrations than their lean counterparts.

HepG2 cells were incubated for 24 h with 10% serum from human lean and obese individuals. The results demonstrated a striking and significant 5- to 8-fold greater stimulatory effect of obese human serum on cellular apoB protein expression versus lean controls (determined via immunoprecipitation and Western blot of cell lysates. This is the first identification of stimulatory effect of obese human serum on hepatocyte apoB. Serum resistin levels were further measured in all subjects via ELISA, showing a significant 50% elevation in serum resistin levels in obese versus lean individuals, associated with the greater obese serum stimulation of hepatocyte apoB, and implicating elevated serum resistin in obesity with increased hepatocyte apoB production.

Lean serum stimulation of hepatocyte cellular apoB expression (24 hours) was then compared with serum-free incubation of hepatocytes apoB expression and apoB expression was found to be 30% lower with lean serum stimulation of hepatocytes versus serum-free controls. To further determine whether resistin in human serum directly plays a quantitatively important role in mediating hepatocyte apoB production, polyclonal antibody removal of serum resistin was performed, and the subsequent effect on cellular apoB expression determined.

Antibody removal of resistin in lean human serum diminished cellular apoB significantly and remarkably by 50%; antibody removal of resistin in obese serum significantly reduced cellular apoB by 30%. These results indicate that resistin in human serum plays a quantitatively important role in mediating hepatocyte apoB production. This further indicates that reduction or inhibition of serum resistin in humans is an effective treatment for hepatic VLDL overproduction and dyslipidemia, both in obese and non-obese states.

### Example 4

### Effect of Resistin siRNA on Cellular Levels of LDL Receptors, PCSK9 and Apo B

HepG2 cells were seeded 200,000 cells per well in a 6-well plate in 2300 uL of 10% FBS DMEM with antibiotics. Awaiting transfection, the cells were placed at 37° C. and 5% CO2. Next, 300 ng of siRNA against RETN (human resistin gene), with the sequence 5′CCCTAATATTTAGGGCAATAA (SEQ ID NO: 5), purchased from Qiagen, MD, was diluted in 100 uL of serum free DMEM which yielded a final concentration of 10 nM once added to cells. 12 uL of HiPerfect Transfection reagent (Qiagen) was also added to the siRNA and mixed by pipetting. The siRNA mixture was allowed to form transfection complexes by incubation at room temperature for 7 minutes. The complexes were then added drop wise to the cells and incubated at 37° C. and 5% CO2 for 24 or 48 hours before harvest. Transfection efficiency was quantitatively measured by the fluorescently labeled scrambled negative control siRNA (Qiagen). siRNA knockdown efficiency was measured by positive control siGAPDH (Qigen) at the transcript and protein levels.

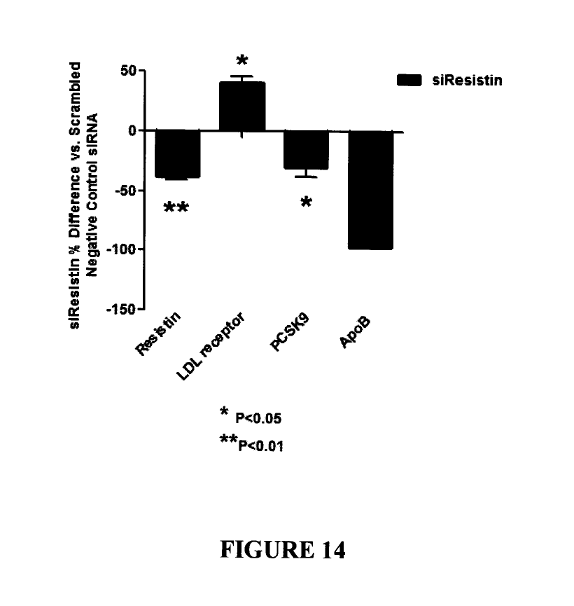

Resistin siRNA was found to be very effective in reducing cellular protein levels of resistin and reducing the expression of apoB (the major protein in cell-produced atherogenic VLDL and LDL particles) as shown in FIG. 14. Resistin siRNA was also very effective in increasing cellular LDL receptor levels, mediated by a reduction in cellular PCSK9 levels. This effect of resistin siRNA in raising cellular LDL receptor levels can enhance hepatocyte uptake and liver clearance of circulating LDL particles, thereby reducing serum levels of LDL particles and LDL-cholesterol.

We claim:

1. A method of modulating the level of lipoproteins in human cells comprising the step of inhibiting resistin within the cellular environment.

2. The method of claim 1, wherein resistin expression is inhibited.

3. The method of claim 1, wherein resistin activity is inhibited.

4. The method of claim 1, wherein the lipoproteins are selected from the group consisting of very low density lipoproteins (VLDL), low density lipoproteins (LDL), intermediate-density lipoproteins (IDL), apolipoproteins, cholesterol and triglycerides.

5. The method of claim 1, wherein resistin is inhibited to achieve a lipoprotein level that is typical of human subject having a body mass index (BMI) of less than or equal to about 25 kg/m².

6. The method of claim 1, wherein resistin is inhibited immunologically.

7. The method of claim 1, wherein resistin is inhibited by a polynucleotide derived from SEQ ID NO: 1.

8. The method of claim 7, wherein the polynucleotide is siRNA.

9. The method of claim 1 to treat a pathological condition in a human subject that results from increased serum levels of undesirable lipoproteins.

10. The method of claim 9, wherein the pathological condition is selected from the group consisting of dyslipidemia, atherosclerosis, coronary heart disease, myocardial infarction, stroke, venous thromboembolism, arterial thromboembolism, obesity, ischemia, stenosis, angina, diabetes and glucose dysregulation.

11. The method of claim 9, wherein the subject has a level of at least about 90 mg/dL of serum apoB, 150 mg/dL of serum triglycerides, 130 mg/dL of LDL cholesterol or 180 mg/dL of serum cholesterol.

12. The method of claim 9, including the step of administering to the subject a statin.

13. A method of screening candidate resistin inhibiting compounds comprising the steps of:
a) incubating a candidate compound with resistin-expressing sample; and
b) measuring the activity of resistin in the sample, wherein a reduction in the activity of resistin in the sample in comparison to a control value obtained in the absence of incubation with the candidate indicates that the candidate compound is a resistin inhibitor.

14. The method of claim 13, wherein resistin activity is determined by measuring the level of resistin mRNA, resistin, LDL receptor, PCSK9, VLDL, LDL, apolipoprotein, cholesterol or triglycerides.

15. A method of diagnosing elevated lipoprotein levels in a human subject comprising the step of determining in a resistin-expressing sample obtained from the subject the level or activity of resistin, PCSK9 or MTP, wherein an increase in the level or activity of resistin, PCSK9 or MTP in comparison to a control level is indicative of elevated lipoprotein levels.

16. The method of claim 15, wherein the level or activity of PCSK9 is determined.

17. The method of claim 15, wherein the level or activity of MTP is determined.
